SEQUENCE LISTING (105 sequences)

SEQ ID NO: 1 - DNA, 50 bp, synthetic construct
  source 1..50: mol_type = other DNA; note = Description of Artificial Sequence: Synthetic oligonucleotide; note = The sequence was designed and synthesized
taaacaaacrtaaaatctataatctcccaacacttaaaaacraatctaac

SEQ ID NO: 2 - DNA, 50 bp, synthetic construct
  source 1..50: mol_type = other DNA; note = Description of Artificial Sequence: Synthetic oligonucleotide; note = The sequence was designed and synthesized
aacaaataactaaaataaaaaaatccaatcaaattccaactccaaaaacc

SEQ ID NO: 3 - DNA, 50 bp, synthetic construct
  source 1..50: mol_type = other DNA; note = Description of Artificial Sequence: Synthetic oligonucleotide; note = The sequence was designed and synthesized
attaactaaaatcataacaaataaccaatcaatattttccatctaactcc

SEQ ID NO: 4 - DNA, 50 bp, synthetic construct
  source 1..50: mol_type = other DNA; note = Description of Artificial Sequence: Synthetic oligonucleotide; note = The sequence was designed and synthesized
aaccccraacaaccacacaacaaaattctatatttcaaaataaaaactcc

SEQ ID NO: 5 - DNA, 50 bp, synthetic construct
  source 1..50: mol_type = other DNA; note = Description of Artificial Sequence: Synthetic oligonucleotide; note = The sequence was designed and synthesized
tcaaaaaacaacttataattctctaatctcctactctaaaccraaaaaac

SEQ ID NO: 6 - DNA, 50 bp, synthetic construct
  source 1..50: mol_type = other DNA; note = Description of Artificial Sequence: Synthetic oligonucleotide; note = The sequence was designed and synthesized
ctaaaaaaacaaaatttaaattcatcaccatatccccaatacctaacaca

SEQ ID NO: 7 - DNA, 50 bp, synthetic construct
  source 1..50: mol_type = other DNA; note = Description of Artificial Sequence: Synthetic oligonucleotide; note = The sequence was designed and synthesized
ttcctaaacctatattaactaatcacaatctactaactctcataattaac

SEQ ID NO: 8 - DNA, 50 bp, synthetic construct
  source 1..50: mol_type = other DNA; note = Description of Artificial Sequence: Synthetic oligonucleotide; note = The sequence was designed and synthesized
catcaacactttatacctacaacrtaaaaacacacaaaatcaccttctac

SEQ ID NO: 9 - DNA, 50 bp, synthetic construct
  source 1..50: mol_type = other DNA; note = Description of Artificial Sequence: Synthetic oligonucleotide; note = The sequence was designed and synthesized
cacaacactaccaacaacacaaataataaactatatacaattcaccacca

SEQ ID NO: 10 - DNA, 50 bp, synthetic construct
  source 1..50: mol_type = other DNA; note = Description of Artificial Sequence: Synthetic oligonucleotide; note = The sequence was designed and synthesized
taacaacttcrccctccataacccttctataatatttttattttctcttc

SEQ ID NO: 11 - DNA, 50 bp, synthetic construct
  source 1..50: mol_type = other DNA; note = Description of Artificial Sequence: Synthetic oligonucleotide; note = The sequence was designed and synthesized
aaaaaaacaacaccaaacaactccctctaaccaaaactaaaaataataca

SEQ ID NO: 12 - DNA, 50 bp, synthetic construct
  source 1..50: mol_type = other DNA; note = Description of Artificial Sequence: Synthetic oligonucleotide; note = The sequence was designed and synthesized
caaccctctacctcctcccaaaaaaatctacaaacttcatatcctttaca

SEQ ID NO: 13 - DNA, 50 bp, synthetic construct
  source 1..50: mol_type = other DNA; note = Description of Artificial Sequence: Synthetic oligonucleotide; note = The sequence was designed and synthesized
acaatctatctaaatattaaccaaaaaaatccaaactctttctaaatctc

SEQ ID NO: 14 - DNA, 50 bp, synthetic construct
  source 1..50: mol_type = other DNA; note = Description of Artificial Sequence: Synthetic oligonucleotide; note = The sequence was designed and synthesized
cttacttaacctatatacattactaaaaaaaataaacataaacctaaaca

SEQ ID NO: 15 - DNA, 50 bp, synthetic construct
  source 1..50: mol_type = other DNA; note = Description of Artificial Sequence: Synthetic oligonucleotide; note = The sequence was designed and synthesized
cccaaaactatccccaacaatcaaataaaaacataattaactataaaaca

SEQ ID NO: 16 - DNA, 50 bp, synthetic construct
  source 1..50: mol_type = other DNA; note = Description of Artificial Sequence: Synthetic oligonucleotide; note = The sequence was designed and synthesized
aaaaacactataactatcattccttcttctacaaacaaataaaaaaaaca

SEQ ID NO: 17 - DNA, 50 bp, synthetic construct
  source 1..50: mol_type = other DNA; note = Description of Artificial Sequence: Synthetic oligonucleotide; note = The sequence was designed and synthesized
attctactcaatacacacaataaacacctacatactaactaaccaaaaca

SEQ ID NO: 18 - DNA, 50 bp, synthetic construct
  source 1..50: mol_type = other DNA; note = Description of Artificial Sequence: Synthetic oligonucleotide; note = The sequence was designed and synthesized
tattctctaaaaactcaatattacaaaaatacaacccctctcccctctca

SEQ ID NO: 19 - DNA, 50 bp, synthetic construct
  source 1..50: mol_type = other DNA; note = Description of Artificial Sequence: Synthetic oligonucleotide; note = The sequence was designed and synthesized
acaaaatttaattctttaaccrctttataaaacctctatctatatccaac

SEQ ID NO: 20 - DNA, 50 bp, synthetic construct
  source 1..50: mol_type = other DNA; note = Description of Artificial Sequence: Synthetic oligonucleotide; note = The sequence was designed and synthesized
acataaaaaataacaaaataaataaaaaccccacactaaccccaattcca

SEQ ID NO: 21 - DNA, 50 bp, synthetic construct
  source 1..50: mol_type = other DNA; note = Description of Artificial Sequence: Synthetic oligonucleotide; note = The sequence was designed and synthesized
taaaactcataaaaacaaaccccaatacctaaacaacacctcaaaaccca

SEQ ID NO: 22 - DNA, 50 bp, synthetic construct
  source 1..50: mol_type = other DNA; note = Description of Artificial Sequence: Synthetic oligonucleotide; note = The sequence was designed and synthesized
caaaaaataaaaccaaaccaacaacccctacctccaaaaaaaccctaaca

SEQ ID NO: 23 - DNA, 50 bp, synthetic construct
  source 1..50: mol_type = other DNA; note = Description of Artificial Sequence: Synthetic oligonucleotide; note = The sequence was designed and synthesized
tttcatcatttcattaaaaattaaaaccctaaatcaacattccccaacca

SEQ ID NO: 24 - DNA, 50 bp, synthetic construct
  source 1..50: mol_type = other DNA; note = Description of Artificial Sequence: Synthetic oligonucleotide; note = The sequence was designed and synthesized
cctaacccataacttccaaacctaaaaaatttaataacacataaaaacca

SEQ ID NO: 25 - DNA, 50 bp, synthetic construct
  source 1..50: mol_type = other DNA; note = Description of Artificial Sequence: Synthetic oligonucleotide; note = The sequence was designed and synthesized
taactataactctaaaataataccccataatccccatcaaacaaataaca

SEQ ID NO: 26 - DNA, 50 bp, synthetic construct
  source 1..50: mol_type = other DNA; note = Description of Artificial Sequence: Synthetic oligonucleotide; note = The sequence was designed and synthesized
aaaaaccaccaaacaaaatcaaacccacaaaaacaaactactaaaaatca

SEQ ID NO: 27 - DNA, 50 bp, synthetic construct
  source 1..50: mol_type = other DNA; note = Description of Artificial Sequence: Synthetic oligonucleotide; note = The sequence was designed and synthesized
aataacaaatacacacacaccactatcaaaacaaaaacaaaatacaaaca

SEQ ID NO: 28 - DNA, 50 bp, synthetic construct
  source 1..50: mol_type = other DNA; note = Description of Artificial Sequence: Synthetic oligonucleotide; note = The sequence was designed and synthesized
aaaacrcctcttttcttccttaacattaattaactcaaattcrttaaaac

SEQ ID NO: 29 - DNA, 50 bp, synthetic construct
  source 1..50: mol_type = other DNA; note = Description of Artificial Sequence: Synthetic oligonucleotide; note = The sequence was designed and synthesized
acaaaaaaacccaaaaactaacaaccaaaaacataataaaacaaacccca

SEQ ID NO: 30 - DNA, 50 bp, synthetic construct
  source 1..50: mol_type = other DNA; note = Description of Artificial Sequence: Synthetic oligonucleotide; note = The sequence was designed and synthesized
caacaaacactacaaaaaaaatataacaaacaaaaacatccaaaccaaca

SEQ ID NO: 31 - DNA, 50 bp, synthetic construct
  source 1..50: mol_type = other DNA; note = Description of Artificial Sequence: Synthetic oligonucleotide; note = The sequence was designed and synthesized
ttcccatacaatcaaaaatccaaaaacattaaaactcaacaaatcaaaca

SEQ ID NO: 32 - DNA, 50 bp, synthetic construct
  source 1..50: mol_type = other DNA; note = Description of Artificial Sequence: Synthetic oligonucleotide; note = The sequence was designed and synthesized
aacaaaatcaaacccacaaaaacaaactactaaaaatcacaaaatttaca

SEQ ID NO: 33 - DNA, 50 bp, synthetic construct
  source 1..50: mol_type = other DNA; note = Description of Artificial Sequence: Synthetic oligonucleotide; note = The sequence was designed and synthesized
aaacaaaacaacactaaaccaaacactaatattcctaaccaaaacaaaca

SEQ ID NO: 34 - DNA, 50 bp, synthetic construct
  source 1..50: mol_type = other DNA; note = Description of Artificial Sequence: Synthetic oligonucleotide; note = The sequence was designed and synthesized
ractctatacaaataaaaaaacratacctacatactaataaactaaaccc

SEQ ID NO: 35 - DNA, 50 bp, synthetic construct
  source 1..50: mol_type = other DNA; note = Description of Artificial Sequence: Synthetic oligonucleotide; note = The sequence was designed and synthesized
taaaaaatcaatttttaataactccctaaaaacacacttcataaaaatac

SEQ ID NO: 36 - DNA, 50 bp, synthetic construct
  source 1..50: mol_type = other DNA; note = Description of Artificial Sequence: Synthetic oligonucleotide; note = The sequence was designed and synthesized
aaatattcaaactacaaaaccttcaacaaacraaacttatacaaataaac

SEQ ID NO: 37 - DNA, 50 bp, synthetic construct
  source 1..50: mol_type = other DNA; note = Description of Artificial Sequence: Synthetic oligonucleotide; note = The sequence was designed and synthesized
cctcattctactaaccaataaccacaataataccataaataaacttatca

SEQ ID NO: 38 - DNA, 50 bp, synthetic construct
  source 1..50: mol_type = other DNA; note = Description of Artificial Sequence: Synthetic oligonucleotide; note = The sequence was designed and synthesized
cttttaaaatcccccaccactaatccccctacaaaaaaccraaaaaatcc

SEQ ID NO: 39 - DNA, 50 bp, synthetic construct
  source 1..50: mol_type = other DNA; note = Description of Artificial Sequence: Synthetic oligonucleotide; note = The sequence was designed and synthesized
aatcacctcaccaaaccctaaattaaaacaaaaaacaaaacatatcaaca

SEQ ID NO: 40 - DNA, 50 bp, synthetic construct
  source 1..50: mol_type = other DNA; note = Description of Artificial Sequence: Synthetic oligonucleotide; note = The sequence was designed and synthesized
aatcacatataattcattacaaaccctcccaacaaaaaaaaattaaacca

SEQ ID NO: 41 - DNA, 50 bp, synthetic construct
  source 1..50: mol_type = other DNA; note = Description of Artificial Sequence: Synthetic oligonucleotide; note = The sequence was designed and synthesized
tccaaccaacaaaaactaaaaaaccacaactacaactccataaataaaca

SEQ ID NO: 42 - DNA, 50 bp, synthetic construct
  source 1..50: mol_type = other DNA; note = Description of Artificial Sequence: Synthetic oligonucleotide; note = The sequence was designed and synthesized
aaaaacaacaaaaactcaatactacrcaataataattaaaaaacrctaac

SEQ ID NO: 43 - DNA, 50 bp, synthetic construct
  source 1..50: mol_type = other DNA; note = Description of Artificial Sequence: Synthetic oligonucleotide; note = The sequence was designed and synthesized
aaaaccactaaaaacaaacataaaaaaaacaaataccactacttcccaca

SEQ ID NO: 44 - DNA, 50 bp, synthetic construct
  source 1..50: mol_type = other DNA; note = Description of Artificial Sequence: Synthetic oligonucleotide; note = The sequence was designed and synthesized
ttttccacttcatacrccrattctaaaaccaaatcttaatctatctttcc

SEQ ID NO: 45 - DNA, 50 bp, synthetic construct
  source 1..50: mol_type = other DNA; note = Description of Artificial Sequence: Synthetic oligonucleotide; note = The sequence was designed and synthesized
aatcaatactaacatccaaattaacaaccaaaaaacaaacaatacacaca

SEQ ID NO: 46 - DNA, 50 bp, synthetic construct
  source 1..50: mol_type = other DNA; note = Description of Artificial Sequence: Synthetic oligonucleotide; note = The sequence was designed and synthesized
acaaatccaactcatcaatattttaaaaaataacccaaccttaaaataca

SEQ ID NO: 47 - DNA, 50 bp, synthetic construct
  source 1..50: mol_type = other DNA; note = Description of Artificial Sequence: Synthetic oligonucleotide; note = The sequence was designed and synthesized
accaaatcttaatctatctttccataaaacaaaacatatacacaatctca

SEQ ID NO: 48 - DNA, 50 bp, synthetic construct
  source 1..50: mol_type = other DNA; note = Description of Artificial Sequence: Synthetic oligonucleotide; note = The sequence was designed and synthesized
aaaatcaaatcaaaattaacaaacaaatccaaaacctacatccaaaaaca

SEQ ID NO: 49 - DNA, 50 bp, synthetic construct
  source 1..50: mol_type = other DNA; note = Description of Artificial Sequence: Synthetic oligonucleotide; note = The sequence was designed and synthesized
actaacacaataaaaccaaacacaactaaacaacaaaaaactaaaccaca

SEQ ID NO: 50 - DNA, 50 bp, synthetic construct
  source 1..50: mol_type = other DNA; note = Description of Artificial Sequence: Synthetic oligonucleotide; note = The sequence was designed and synthesized
aacactaatacaactactcttcaaaaccataaaacacaaaaacaaaaaca

SEQ ID NO: 51 - DNA, 50 bp, synthetic construct
  source 1..50: mol_type = other DNA; note = Description of Artificial Sequence: Synthetic oligonucleotide; note = The sequence was designed and synthesized
aaaacaaaaactactcttaaccttaactcccaacctaaaaaataaaaacc

SEQ ID NO: 52 - DNA, 50 bp, synthetic construct
  source 1..50: mol_type = other DNA; note = Description of Artificial Sequence: Synthetic oligonucleotide; note = The sequence was designed and synthesized
attcaatcatcccaaaacatctaaaaacaaacaaataccaaaatacacac

SEQ ID NO: 53 - DNA, 50 bp, synthetic construct
  source 1..50: mol_type = other DNA; note = Description of Artificial Sequence: Synthetic oligonucleotide; note = The sequence was designed and synthesized
tccctaaatcaacataatacataatatataaataaactcttaattcaatc

SEQ ID NO: 54 - DNA, 50 bp, synthetic construct
  source 1..50: mol_type = other DNA; note = Description of Artificial Sequence: Synthetic oligonucleotide; note = The sequence was designed and synthesized
tctccatattttttcccacrtactatctttactttctattttcttaattc

SEQ ID NO: 55 - DNA, 50 bp, synthetic construct
  source 1..50: mol_type = other DNA; note = Description of Artificial Sequence: Synthetic oligonucleotide; note = The sequence was designed and synthesized
caaacaattcccacccaaccaacaaacaaaaacaactacaacctaaatac

SEQ ID NO: 56 - DNA, 50 bp, synthetic construct
  source 1..50: mol_type = other DNA; note = Description of Artificial Sequence: Synthetic oligonucleotide; note = The sequence was designed and synthesized
ctcatccctaaaacactataacaatccaacrtaaataactaaaataaacc

SEQ ID NO: 57 - DNA, 50 bp, synthetic construct
  source 1..50: mol_type = other DNA; note = Description of Artificial Sequence: Synthetic oligonucleotide; note = The sequence was designed and synthesized
ctatctataaacctacttccaaaccattcaaaatatacatcaaattactc

SEQ ID NO: 58 - DNA, 50 bp, synthetic construct
  source 1..50: mol_type = other DNA; note = Description of Artificial Sequence: Synthetic oligonucleotide; note = The sequence was designed and synthesized
aacaaaaactaacaaaaacaaactttctaataataaacaaaactatacca

SEQ ID NO: 59 - DNA, 50 bp, synthetic construct
  source 1..50: mol_type = other DNA; note = Description of Artificial Sequence: Synthetic oligonucleotide; note = The sequence was designed and synthesized
ctataaacccaaaatcctcaaaccataccaacaaattaactataaaccca

SEQ ID NO: 60 - DNA, 50 bp, synthetic construct
  source 1..50: mol_type = other DNA; note = Description of Artificial Sequence: Synthetic oligonucleotide; note = The sequence was designed and synthesized
tctacracctcttcctactacaaataacaaccatcttaaacataataccc

SEQ ID NO: 61 - DNA, 50 bp, synthetic construct
  source 1..50: mol_type = other DNA; note = Description of Artificial Sequence: Synthetic oligonucleotide; note = The sequence was designed and synthesized
tcctaaaactacatttaaataacccaattcctcaactttaactttaaaac

SEQ ID NO: 62 - DNA, 50 bp, synthetic construct
  source 1..50: mol_type = other DNA; note = Description of Artificial Sequence: Synthetic oligonucleotide; note = The sequence was designed and synthesized
caaattaatatttttcatactaacaaaaaataactactataacttttccc

SEQ ID NO: 63 - DNA, 50 bp, synthetic construct
  source 1..50: mol_type = other DNA; note = Description of Artificial Sequence: Synthetic oligonucleotide; note = The sequence was designed and synthesized
aactttataaacaataaacaaaaaaacacaaaaacaaaaaccctaacaca

SEQ ID NO: 64 - DNA, 50 bp, synthetic construct
  source 1..50: mol_type = other DNA; note = Description of Artificial Sequence: Synthetic oligonucleotide; note = The sequence was designed and synthesized
ttaaaaacatcaaccaccataaccacataatcctaaaaattaaaaaccca

SEQ ID NO: 65 - DNA, 50 bp, synthetic construct
  source 1..50: mol_type = other DNA; note = Description of Artificial Sequence: Synthetic oligonucleotide; note = The sequence was designed and synthesized
aaacacaactctaaaaacattataccttaattttctaattcratttcaac

SEQ ID NO: 66 - DNA, 50 bp, synthetic construct
  source 1..50: mol_type = other DNA; note = Description of Artificial Sequence: Synthetic oligonucleotide; note = The sequence was designed and synthesized
aacaatttaatcctcaaaaacaaaactcaaccaataaccaaacaaactac

SEQ ID NO: 67 - DNA, 50 bp, synthetic construct
  source 1..50: mol_type = other DNA; note = Description of Artificial Sequence: Synthetic oligonucleotide; note = The sequence was designed and synthesized
taacaaaaataaacatcataccaattaacaacttccaattttacctcttc

SEQ ID NO: 68 - DNA, 50 bp, synthetic construct
  source 1..50: mol_type = other DNA; note = Description of Artificial Sequence: Synthetic oligonucleotide; note = The sequence was designed and synthesized
accacaataaccaacaccctcaaaataaacaaaaatataatactaaccca

SEQ ID NO: 69 - DNA, 51 bp, synthetic construct
  source 1..51: mol_type = other DNA; note = Description of Artificial Sequence: Synthetic oligonucleotide; note = The sequence was designed and synthesized
aaaatacaatacaaccaaaacccaaatacaaattacaaataaatacacaca

SEQ ID NO: 70 - DNA, 50 bp, synthetic construct
  source 1..50: mol_type = other DNA; note = Description of Artificial Sequence: Synthetic oligonucleotide; note = The sequence was designed and synthesized
acctaccaaaaaaaataactctacatactataaaacaaaaaactaaatcc

SEQ ID NO: 71 - DNA, 50 bp, synthetic construct
  source 1..50: mol_type = other DNA; note = Description of Artificial Sequence: Synthetic oligonucleotide; note = The sequence was designed and synthesized
aaattaatttcccctactatattactaaaaaataaacacracatatctac

SEQ ID NO: 72 - DNA, 50 bp, synthetic construct
  source 1..50: mol_type = other DNA; note = Description of Artificial Sequence: Synthetic oligonucleotide; note = The sequence was designed and synthesized
aaaaaaaactccataccaattatatacaaataacctacacaacccactca

SEQ ID NO: 73 - DNA, 50 bp, synthetic construct
  source 1..50: mol_type = other DNA; note = Description of Artificial Sequence: Synthetic oligonucleotide; note = The sequence was designed and synthesized
aataattaaaaatcataacctaccccccctctaaaacatacaccttaaac

SEQ ID NO: 74 - DNA, 50 bp, synthetic construct
  source 1..50: mol_type = other DNA; note = Description of Artificial Sequence: Synthetic oligonucleotide; note = The sequence was designed and synthesized
atacaattttttatcaaaaccaaataacataacaatacatacaaaacttc

SEQ ID NO: 75 - DNA, 50 bp, synthetic construct
  source 1..50: mol_type = other DNA; note = Description of Artificial Sequence: Synthetic oligonucleotide; note = The sequence was designed and synthesized
ttatattaaaaaacrtaacctaataacatcacaactaaccraacaaccrc

SEQ ID NO: 76 - DNA, 50 bp, synthetic construct
  source 1..50: mol_type = other DNA; note = Description of Artificial Sequence: Synthetic oligonucleotide; note = The sequence was designed and synthesized
aaacaccactccctcaaactaccacatatattattaaaaaacataaaaca

SEQ ID NO: 77 - DNA, 50 bp, synthetic construct
  source 1..50: mol_type = other DNA; note = Description of Artificial Sequence: Synthetic oligonucleotide; note = The sequence was designed and synthesized
acaaaaaccccaacctaacaacccacaaaaaaatacccaaaaaacaatca

SEQ ID NO: 78 - DNA, 50 bp, synthetic construct
  source 1..50: mol_type = other DNA; note = Description of Artificial Sequence: Synthetic oligonucleotide; note = The sequence was designed and synthesized
tttcccaatctatccactccactctatttctaatacctaaatcataattc

SEQ ID NO: 79 - DNA, 50 bp, synthetic construct
  source 1..50: mol_type = other DNA; note = Description of Artificial Sequence: Synthetic oligonucleotide; note = The sequence was designed and synthesized
caaaataaaattccaacccaaatcttccactaatccaaatctataaactc

SEQ ID NO: 80 - DNA, 50 bp, synthetic construct
  source 1..50: mol_type = other DNA; note = Description of Artificial Sequence: Synthetic oligonucleotide; note = The sequence was designed and synthesized
aaataaatcatcacactataatccactaccaaataataattctaaatccc

SEQ ID NO: 81 - DNA, 50 bp, synthetic construct
  source 1..50: mol_type = other DNA; note = Description of Artificial Sequence: Synthetic oligonucleotide; note = The sequence was designed and synthesized
aataaatcatccaaaataaaataaaaacacaaaccttacaacccaataca

SEQ ID NO: 82 - DNA, 50 bp, synthetic construct
  source 1..50: mol_type = other DNA; note = Description of Artificial Sequence: Synthetic oligonucleotide; note = The sequence was designed and synthesized
cctaaatcaaaattactatcactataactttcaattaacccracrtaaac

SEQ ID NO: 83 - DNA, 50 bp, synthetic construct
  source 1..50: mol_type = other DNA; note = Description of Artificial Sequence: Synthetic oligonucleotide; note = The sequence was designed and synthesized
taaaaacaaactccaaaacctaactacaaaataaatatccaaaacactca

SEQ ID NO: 84 - DNA, 50 bp, synthetic construct
  source 1..50: mol_type = other DNA; note = Description of Artificial Sequence: Synthetic oligonucleotide; note = The sequence was designed and synthesized
aaacttttatttatttaccractcctacacataactactaaaactcaaac

SEQ ID NO: 85 - DNA, 50 bp, synthetic construct
  source 1..50: mol_type = other DNA; note = Description of Artificial Sequence: Synthetic oligonucleotide; note = The sequence was designed and synthesized
aactaccatctaaacctaaacccatctaatatacataacccataattaac

SEQ ID NO: 86 - DNA, 50 bp, synthetic construct
  source 1..50: mol_type = other DNA; note = Description of Artificial Sequence: Synthetic oligonucleotide; note = The sequence was designed and synthesized
cccatatcataaaaacatcacraaaatactaacctaaaccaaaaaacacc

SEQ ID NO: 87 - DNA, 50 bp, synthetic construct
  source 1..50: mol_type = other DNA; note = Description of Artificial Sequence: Synthetic oligonucleotide; note = The sequence was designed and synthesized
taaaactttatatactctcataaacratctctttaatattctaacatccc

SEQ ID NO: 88 - DNA, 50 bp, synthetic construct
  source 1..50: mol_type = other DNA; note = Description of Artificial Sequence: Synthetic oligonucleotide; note = The sequence was designed and synthesized
caaccccrtaaccttacrtacaaaaaacaattttcatatttccaaaatac

SEQ ID NO: 89 - DNA, 50 bp, synthetic construct
  source 1..50: mol_type = other DNA; note = Description of Artificial Sequence: Synthetic oligonucleotide; note = The sequence was designed and synthesized
crataccaactacctttttattcctccccacctctaacttatctactaac

SEQ ID NO: 90 - DNA, 50 bp, synthetic construct
  source 1..50: mol_type = other DNA; note = Description of Artificial Sequence: Synthetic oligonucleotide; note = The sequence was designed and synthesized
rtacctaataataattataaaaattcctctactacaaaaaaacracracc

SEQ ID NO: 91 - DNA, 50 bp, synthetic construct
  source 1..50: mol_type = other DNA; note = Description of Artificial Sequence: Synthetic oligonucleotide; note = The sequence was designed and synthesized
taaaaccaactattaattatttatcataacaaaaacaaaaataacctatc

SEQ ID NO: 92 - DNA, 50 bp, synthetic construct
  source 1..50: mol_type = other DNA; note = Description of Artificial Sequence: Synthetic oligonucleotide; note = The sequence was designed and synthesized
actcaaacaaacctactacctacrcctactaaataacaaaaactacaaac

SEQ ID NO: 93 - DNA, 50 bp, synthetic construct
  source 1..50: mol_type = other DNA; note = Description of Artificial Sequence: Synthetic oligonucleotide; note = The sequence was designed and synthesized
aaataattataaactattaaaatataaaccaataattcccaaactctaac

SEQ ID NO: 94 - DNA, 50 bp, synthetic construct
  source 1..50: mol_type = other DNA; note = Description of Artificial Sequence: Synthetic oligonucleotide; note = The sequence was designed and synthesized
cctcttccctcctttaacttcaaattttatttttccctatctacacrcac

SEQ ID NO: 95 - DNA, 50 bp, synthetic construct
  source 1..50: mol_type = other DNA; note = Description of Artificial Sequence: Synthetic oligonucleotide; note = The sequence was designed and synthesized
cctatattcaaaacctaaacaaccctccrtaataaaataaaaacactatc

SEQ ID NO: 96 - DNA, 50 bp, synthetic construct
  source 1..50: mol_type = other DNA; note = Description of Artificial Sequence: Synthetic oligonucleotide; note = The sequence was designed and synthesized
ccatttacctaaaaataaaactataaaaaccaccaaaaaaatacctatcc

SEQ ID NO: 97 - DNA, 50 bp, synthetic construct
  source 1..50: mol_type = other DNA; note = Description of Artificial Sequence: Synthetic oligonucleotide; note = The sequence was designed and synthesized
atcccaaatataaactaaaaacacaaaccatccccaatttcccactcttc

SEQ ID NO: 98 - DNA, 50 bp, synthetic construct
  source 1..50: mol_type = other DNA; note = Description of Artificial Sequence: Synthetic oligonucleotide; note = The sequence was designed and synthesized
accctttacctataaaactatatctccatctcaaaaacctatttaaaacc

SEQ ID NO: 99 - DNA, 50 bp, synthetic construct
  source 1..50: mol_type = other DNA; note = Description of Artificial Sequence: Synthetic oligonucleotide; note = The sequence was designed and synthesized
aaaatacataattaactcctaaacctaaaacracctcaaaatactacaac

SEQ ID NO: 100 - DNA, 50 bp, synthetic construct
  source 1..50: mol_type = other DNA; note = Description of Artificial Sequence: Synthetic oligonucleotide; note = The sequence was designed and synthesized
accatacacccctaacaacatcaactttcaaaatattcctaaaacacaca

SEQ ID NO: 101 - DNA, 101 bp, synthetic construct
  source 1..101: mol_type = other DNA; note = Description of Artificial Sequence: Synthetic polynucleotide; note = The sequence was designed and synthesized
gcactacagccagtcaccagcaatgactgcaagtaactctaggacactgacgcctatttgatttggaagagaataaggaacataatgatgcctgaaatgtc

SEQ ID NO: 102 - DNA, 101 bp, synthetic construct
  source 1..101: mol_type = other DNA; note = Description of Artificial Sequence: Synthetic polynucleotide; note = The sequence was designed and synthesized
gacatttcaggcatcattatgttccttattctcttccaaatcaaataggcgtcagtgtcctagagttacttgcagtcattgctggtgactggctgtagtgc

SEQ ID NO: 103 - DNA, 101 bp, synthetic construct; identical to SEQ ID NO: 102
  source 1..101: mol_type = other DNA; note = Description of Artificial Sequence: Synthetic polynucleotide; note = The sequence was designed and synthesized

SEQ ID NO: 104 - DNA, 101 bp, synthetic construct; identical to SEQ ID NO: 101
  source 1..101: mol_type = other DNA; note = Description of Artificial Sequence: Synthetic polynucleotide; note = The sequence was designed and synthesized

SEQ ID NO: 105 - DNA, 101 bp, synthetic construct; identical to SEQ ID NO: 101
  source 1..101: mol_type = other DNA; note = Description of Artificial Sequence: Synthetic polynucleotide; note = The sequence was designed and synthesized

(19) United States
(12) Patent Application Publication
(10) Pub. No.: US 2026/0226559 A1
(43) Pub. Date: Aug. 6, 2026

(54) METHOD TO MEASURE MYELOID SUPPRESSOR CELLS FOR DIAGNOSIS AND PROGNOSIS OF CANCER

(71) Applicants: BROWN UNIVERSITY, Providence, RI (US); THE REGENTS OF THE UNIVERSITY OF CALIFORNIA, Oakland, CA (US); UNIVERSITY OF KANSAS, Lawrence, KS (US); THE TRUSTEES OF DARTMOUTH COLLEGE, Hanover, NH (US)

(72) Inventors: Karl Kelsey, Brookline, MA (US); John Wiencke, San Francisco, CA (US); Devin Koestler, Kansas City, KS (US); Brock Christensen, West Lebanon, NH (US)

(21) Appl. No.: 19/462,420

(22) Filed: Jan. 28, 2026

(51) Int. Cl. C12Q 1/6886; C12Q 1/6827; C12Q 1/6837; C12Q 1/6881; G06F 17/18; G16H 50/20

(52) U.S. Cl. CPC C12Q 1/6886; C12Q 1/6827; C12Q 1/6837; C12Q 1/6881; G06F 17/18; G16H 50/20; C12Q 2600/118; C12Q 2600/154

(57) ABSTRACT

Ratio of neutrophils to lymphocytes (NLR) is here associated with immune suppression and decreased survival times in multiple solid tumors. Based on immune cell-specific DMRs and validated cell deconvolution algorithms, the NLR in blood from glioma patients was estimated and glioma patients had elevated mdNLR scores compared to controls. The patient mdNLR scores were increased in patients with grade IV tumors compared to grade II/III. High mdNLR scores were associated with shorter survival. Candidate single (myeloid-associated) gene loci that were highly correlated with the mdNLR were identified. Single myeloid differentiation loci provide a simpler and cheaper alternative to the mdNLR, which requires complex array data. Immunomethylomics are useful and more convenient than conventional cell analysis in profiling glioma risk and survival.

### RELATED APPLICATIONS

The present application is a continuation application of U.S. patent application Ser. No. 17/937,087 filed Sep. 30, 2022, which is a continuation application of U.S. patent application Ser. No. 16/345,158 filed Apr. 25, 2019, now abandoned, which is a national phase filing under 35 U.S.C. § 371 of International Application No. PCT/US2017/058470 filed Oct. 26, 2017, which claims the benefit of provisional application Ser. No. 62/413,380 entitled “A method to measure myeloid suppressor cells in human blood and tissues”, filed Oct. 26, 2016 with inventors Karl Kelsey, John Wiencke, Devin Koestler, and Brock Christensen, the entire contents of which are hereby incorporated herein by reference in their entirety.

### GOVERNMENT SUPPORT

This invention was made with government support under grant numbers R01CA056689, P50CA097257, R01CA207110, R01CA052689, R01CA126831, R01CA139020, R25CA112355, R01DE022772, R01CA216265, UL1RR024131 and P30CA082103 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jan. 22, 2026, is named 0312021.00266_SL.xml and is 141,520 bytes in size.

### TECHNICAL FIELD

Methods, compositions and devices are provided for measuring amounts of types of leukocytes and associated epigenetic methylation status in biological samples.

### BACKGROUND

A large number of epidemiologic studies of DNA methylation has been driven by appreciation for a role methylation plays in the development and progression of human diseases and the declining cost of high-throughput technologies for interrogating the genome. See, Koestler D C et al. BMC Bioinformatics 17:120 (2016). Studies investigating the role of DNA methylationin human diseases and exposures are referred to as epigenome-wide association studies (EWAS). See, Rakyan V K et al. Nat Rev Genet 12:529-541 (2011). However, owing to tissue specificity of DNA methylation, comparisons of methylation signatures assessed over heterogenous cell populations have been found to be susceptible to confounding and misinterpreted associations. See, Adalsteinsson B T et al. PLoS One 7: e46705 (2012); Reinius L E et al. PLoS One 7: e41361 (2012); Koestler D C et al. Cancer Epidemiol Biomarkers Prev 21:1293-1302 (2012); Lam L L et al. Proc Natl Acad Sci USA 109 Suppl. 2, 17253-17260 (2012). These issues are a foremost challenge facing EWAS. See, Houseman E A et al. Curr Environ Health Rep 2:145-154 (2015); Michels K B et al. Nat Methods 10:949-955 (2013); Jaffe A E et al. Genome Biol 15: R31 (2014); Liang L et al. Hum Mol Genet 23: R83-R88 (2014). Cellular lineage and somatic differentiation are regulated by epigenetic mechanisms, including DNA methylation. See, Accomando W P et al. Genome Biol 15 (3): R50 (2014); Reinius L E et al. PLoS One 7: e41361 (2012); Khavari D A et al. Cell Cycle Georget. Tex. 9 (19): 3880-3883 (2010); Houseman E A et al. Curr Environ Health Rep 2 (2): 145-154 (2015); Houseman E A et al. BMC Bioinformatics 13:86 (2012); Koestler D C et al. BMC Bioinformatics 17:120 (2016). Thus, the pattern of methylation at phenotypically important CpG regions varies across individual tissues and cell types and specifically across the distinct leukocyte subtypes. See, Accomando W P et al. Genome Biol 15 (3): R50 (2014); Reinius L E et al. PLoS One 7: e41361 (2012); Khavari D A et al. Cell Cycle Georget. Tex. 9 (19): 3880-3883 (2010); Houseman E A et al. Curr Environ Health Rep 2 (2): 145-154 (2015); Houseman E A et al. BMC Bioinformatics 13:86 (2012); Koestler D C et al. BMC Bioinformatics 17:120 (2016). Recent attempts aimed at minimizing the potential for confounding in the analysis of DNA methylation data have prompted researchers to restrict methylation assessment to purified cell populations, for example, CD4+ or CD14+ cells isolated from peripheral blood. See, Reynolds L M et al. Nat Commun 5:5366 (2014); Gunawardhana L P et al. Epigenetics 9:1302-1316 (2014). Although such studies may be less prone to confounding by leukocyte-lineage heterogeneity compared to those involving whole blood DNA methylation assessments, purification of cell populations carrying these markers will not completely eliminate heterogeneity attributable to lineage differences. See, Reinius L E et al. PLoS One 7: e41361 (2012). Other attempts to address the potential for confounding in blood-based DNA methylation data have involved adjusting statistical models with additional terms reflecting the cell composition of study samples using, for example, measurements from complete blood cell counts (CBC) or fluorescence-activated cell sorting (FACS). See, Lam L L et al. Proc Natl Acad Sci USA 109 Suppl. 2, 17253-17260 (2012); Marioni R E et al. Int J Epidemiol 44 (4): 1388-96 (2015).

There is a need to optimize DMR libraries to increase the accuracy of cell mixture deconvolution and provide enhanced discrimination between or among leukocyte subtypes of the immune cell landscape for effective prognosis and/or diagnosis of diseases based on leukocyte subtype methylation profiles from DNA methylation data of biological samples, such as blood and tissues.

### SUMMARY

The invention in general provides methods of selecting a CpG site nucleotide sequence to use as a probe, or a family of probes having plurality of such sequences, that are useful to determine percent composition of various leukocyte subtypes in a biological sample, for example, blood, lymph, serum, plasma, or in a tissue exudate or extract, by analyzing extent of methylation at that site. The invention further provides uses of these sequences to determine by extent of methylation, the proportions of leukocyte subtypes, for example, a neutrophil to lymphocyte ratio (NLR), that can be associated with one or more pathological conditions such as a cancer or inflammation. The probes derived from the sequences are used in devices for such analyses.

An aspect of the invention herein provides an array for determining methylation status of leukocyte subtypes in a biological sample by analyzing methylation of a plurality of CpG dinucleotides in a plurality of genes of the sample, the array having the following characteristics:
a surface having a plurality of oligonucleotide probes with nucleotide sequences selected from at least one of the group of SEQ ID NO: 1-100, each probe attached at an addressable location on the surface, each probe hybridizes to a nucleotide sequence of a methylated form or an unmethylated form of a CpG dinucleotide in a sequence of a gene in the sample. The array in various embodiments is further characterized as having:
at least 5 probes, at least 10 probes, at least 25 probes, at least 50 probes, or at least the full 100 probes; and/or,
additional oligonucleotide probes attached to the array containing CpG dinucleotides that optimally discriminate among leukocyte subtypes according to methylation status of CpG dinucleotides in a gene of the leukocyte type, and/or further having control probes; for example, the additional oligonucleotide probes comprise SEQ ID NOs: 101-105; and/or
the oligonucleotide probes of SEQ ID NOs: 1-100 and/or the additional probes are selected to distinguish CpG methylation profile DNA sequences of at least two leukocyte subtypes selected from the group of: myeloid-derived suppressor cells (MDSCs), granulocytic MDSCs (gMDSCs), mMDSCs, mast cells, basophils, neutrophils, eosinophils, monocytes, natural killer cells (NK), activated NK cells, NKT cells, Th17 T cells, megakaryocytes, erythrocytes, cytotoxic T cells, double positive T cells, T helper cells, Treg cells, and B cells.

An aspect of the invention herein provides a method of using an array to determine proportions in a biological sample of a subject of leukocyte subtypes to prognose and/or diagnose a disease state in the subject, the method having steps of:
analyzing extent of hybridization of patient sample DNA to each of a plurality of oligonucleotide probes, the probes being affixed to at least two surfaces for each of methylated and unmethylated CpG sequences and otherwise identical in nucleotide sequence, the plurality of the nucleotide sequences selected from at least one of the group of SEQ ID NO: 1-100, for determining methylation status of at least one CpG dinucleotide in the DNA of the sample;
comparing methylation status of the plurality of CpG dinucleotides analyzed in the patient sample to a DNA methylation reference library, to determine proportion of each leukocyte type in the sample;
displaying the methylation status of the plurality of hybridized genes in the sample in a graphical representation, thereby generating an image of the methylation profile (methylome) of the leukocyte subtypes in the patient sample; and,
prognosing and/or diagnosing the disease state in the patient associated with the methylation status of CpG sites in leukocyte subtypes, the disease state selected from a cancer, a cardiac condition, inflammation, an autoimmune disease, and infection/sepsis.

In an embodiment of the method, the prognosing and/or diagnosing further includes:
associating the methylation status of CpG sites in specific leukocyte subtypes being above a pre-determined statistical threshold by determining a multivariate proportional hazards ratio equal to or greater than 1.0 as an indicium of a prognosis of an increased risk of death in the patient from the disease or as a diagnosis of the disease; or,
associating the proportions of specific leukocyte subtypes above a pre-determined statistical threshold of a neutrophil to lymphocyte ratio (mdNLR) equal to or greater than 1.0, at least about 2.0 or at least about or greater than 4.0 as an indicium of a prognosis of an increased risk of death in the patient from the disease or as a diagnosis of the disease; or,
associating myeloid derived suppressor cell (MDSC), or gMDSC proportions in the sample as greater than or equal to a pre-determined statistical threshold of a multivariate proportional hazard value equal to or greater than 1.0, greater than 2.0, or at least about or greater than 2.5 as an indicium of a prognosis of an increased risk of death in the patient from the disease or as a diagnosis of the disease.

An aspect of the invention herein provides, in a method of predicting a methylation class membership of leukocytes in a bodily fluid sample of a patient, the methylation class membership corresponding to an epigenetic signature of a plurality of leukocyte subtypes, in which the method includes steps of measuring amounts of DNA methylation in each of a plurality of leukocyte type populations to determine differentially methylated regions (DMRs), ranking leukocyte DMRs for each leukocyte type according to statistical strength of association of each of at least one DMR with each leukocyte type, clustering samples in a training set using a defined number of highest ranked leukocyte DMRs to determine clustering solutions, a clustering solution corresponding to the methylation class membership, and predicting the methylation class membership for the leukocyte subtypes within a testing set by applying the clustering solutions obtained from the training set to highest ranked leukocyte DMRs in the testing set, the predicted methylation class membership being determined by testing association of the predicted methylation class membership with the statistical discriminatory strength of the at least one DMR among the leukocyte subtypes, the improvement having the following steps:
obtaining leukocyte methylation data of the sample using an array containing a plurality of nucleotide sequences each having a CpG site affixed to the array;
identifying statistically predictive subset DNA methylation libraries by scanning candidate sets of putative leukocyte-specific methylation markers to find sets of CpG sites that characterize each of the respective leukocyte subtypes in the sample estimated by a cell mixture deconvolution;
constructing and evolving subset libraries of DMRs consisting of CpG sites differentially methylated among leukocyte subtypes, by iteratively selecting subsets of DMRs at each iteration based on the statistical contribution of each DMR to methylation class membership prediction accuracy;
modifying a probability of selection of the DMRs at each iteration, the probability of selection of a CpG being modified proportional to contribution of the at least one DMR to methylation class membership prediction accuracy; and,
comparing the subset library of the patient DMRs sample to DMRs of a reference-based library of a plurality of control samples from a plurality of normal patients, to obtain a prognosis and/or a diagnosis of a cancer of the patient.

An embodiment of this method is further characterized in that, the array for analyzing proportions of specific leukocyte subtypes in the sample having at least one oligonucleotide selected from the group of nucleotide sequences of SEQ ID NO: 1-100, and the leukocyte subtypes are selected from at least one of: myeloid-derived suppressor cells (MDSCs), granulocytic MDSCs (gMDSCs), mast cells, basophils, neutrophils, eosinophils, monocytes, natural killer cells (NK), megakaryocytes, erythrocytes, cytotoxic T cells, double positive T cells, T helper cells, Treg cells, and B cells.

In another embodiment of this method, the applying the subset library further includes: calculating a multivariate proportional hazards ratio for the sample from the patient to assess the relationship of cancer prognosis and/or diagnosis with methylation status of the leukocyte composition.

In yet another embodiment of this method, the step of comparing further includes obtaining the prognosis and/or diagnosis of cancer by selecting the leukocyte composition methylation status from the group of myeloid-derived suppressor cell (MDSC) methylation status and granulocytic myeloid-derived suppressor cell (gMDSC) methylation status.

In yet another embodiment of this method, selecting the leukocyte composition methylation status from the group of myeloid-derived suppressor cell (MDSC) methylation status and granulocytic myeloid-derived suppressor cell (gMDSC) methylation status further includes calculating the gMDSC multivariate proportional hazards ratio, which as equal to or greater than 1.0 is an indicium of a prognosis of an increased risk of death in the patient from the disease or is a diagnosis of the disease.

In yet another embodiment this method further includes associating the multivariate proportional hazards ratio of at least about 1.0, or at least about 2.0 with an indicium of about a two-fold increase in the risk of death in the patient from the cancer.

Yet another embodiment of this method further includes adjusting the multivariate proportional hazards ratio for tumor histology status, gene mutation status, patient age, patient history, and patient gender status.

Yet another embodiment of this method further includes selecting the CpG sites for inclusion in the statistically predictive subset library those CpG methylation patterns that indicate MDSCs or gMDSCs in the sample.

An aspect of the invention herein provides a method of obtaining selection probabilities of leukocyte differentially methylated regions (DMRs) for inclusion in a statistically predictive subset library of DMRs for predicting leukocyte subtype methylation class membership of leukocytes in a blood sample from a subject for prognosis and/or diagnosis of cancer in the subject, the method including:
constructing a candidate DMR search space to compare mean methylation values among leukocyte subtypes by identifying CpGs that uniquely characterize each leukocyte cell type, and randomly assembling subset DMR libraries with CpGs that uniquely characterize the leukocyte cell subtypes through multiple iterations;
estimating leukocyte cell compositions in the sample using the assembled subset DMR libraries and cell mixture deconvolution, and computing leukocyte subtype ratios from the estimated leukocyte compositions of the sample;
assessing the accuracy of leukocyte cell composition estimates by comparing statistical differences among observed cell compositions obtained by at least one method selected from the group of: fluorescence-activated cell sorting (FACS) and complete blood cell counts (CBC), to predicted cell compositions obtained from cell mixture deconvolution of normal control samples, and implementing an iterative leave-one out procedure to assess individual contributions of each CpG to statistical prediction performance of the methylation class membership of the leukocytes, and further computing a dispersion separability criterion (DSC) score to assess a DMR subset power for discriminating among leukocyte subtypes, to select CpGs, and updating subset DMR library selection probabilities by modifying the CpGs selected using the statistical prediction performance of a relative and of an absolute prediction accuracy of each CpG compared to remaining CpGs in the library, and using the updated probabilities in successive iterations to obtain updated probabilities, resulting statistically predictive subset DNA methylation libraries containing CpGs with the largest selection probabilities for improved accuracy of predicting leukocyte type methylation class membership; and,
fitting the multivariate proportional hazards ratio calculated from the sample to the updated subset DMR libraries thereby prognosing and/or diagnosing cancer in the blood sample from the subject.

In an embodiment of this method, the step of computing leukocyte ratios from the estimated leukocyte cell compositions further includes comparing amounts of at least two different leukocyte subtypes present in the leukocyte cell composition of the sample from the subject.

In an embodiment of this method, the step of fitting the multivariate proportional hazards ratio further includes comparing the hazards ratio to a Kaplan Meier plot of cancer survival data to prognose subject survival probability.

The method in an additional embodiment further includes calculating a neutrophil to lymphocyte ratio (mdNLR) and fitting the multivariate proportional hazards ratio to the mdNLR.

The method in certain embodiments of the updated statistically predictive subset DMR library further includes CpG sites of granulocytic myeloid-derived suppressor cells (gMDSCs) in the sample from the subject.

The statistically predictive subset DMR libraries in certain embodiments of the method further include CpG sites the methylation status of which indicates MDSCs in the sample from the subject. In various embodiments of the method the dispersion separability criterion (DSC) score defined as Db/Dw, such that Db is a measure of dispersion between cell types and Dw is a measure of dispersion within cell types, is implemented to quantify dispersion between leukocyte subtypes and within leukocyte subtypes for a randomly selected DMR subset.

The method in various embodiments diagnoses and/or prognoses the cancer which is at least one selected from glioma, breast cancer, lung cancer, prostate cancer, renal cancer, and head and neck cancer.

An aspect of the invention herein provides a device having at least two surfaces each having an array with oligonucleotide probes of defined sequence each at an addressable location, the sequences selected from at least one of the group of SEQ ID NOs: 101-105. The array in various embodiments contains the probes attached to beads, for example, in wells of a multi-well plate, or the probes attached to solid substrates such as glass plates or slides. The device is used to determine proportions of leukocyte subtypes, for diagnosis and/or prognosis of cancers, for example, the cancer which is at least one selected from glioma, breast cancer, lung cancer, prostate cancer, renal cancer, and head and neck cancer. The leukocyte subtypes include at least one or a plurality of the following: myeloid-derived suppressor cells (MDSCs), granulocytic MDSCs (gMDSCs), mMDSCs, mast cells, basophils, neutrophils, eosinophils, monocytes, natural killer cells (NK), activated NK cells, NKT cells, Th17 T cells, megakaryocytes, erythrocytes, cytotoxic T cells, double positive T cells, T helper cells, Treg cells, and B cells. The array can include additional probes, for example selected from SEQ ID Nos: 1-100 and related probes.

### BRIEF DESCRIPTION OF THE DRAWINGS

FIG. 1 are heats map illustrating differences in CpG methylation sites between myeloid derived suppressor cells (MDSC) cells and normal granulocytes. The data were obtained using arrays having the DNA sequences in the column at the right (SEQ ID Nos: 1-100). The six lanes on the left side are data obtained from isolated gMDSCs from six different subjects and the six lanes on the right are data obtained from isolated normal granulocytes from the same subjects. The dark quadrants (upper right and lower left which are blue in original data) contain data for the CpG sites that are unmethylated, and the light quadrants (upper left and lower right which are yellow in original data), the data for the unmethylated CpG sites. This unsupervised cluster analysis demonstrates that the degree of methylation differs dramatically between the two specific cell sub-types, and that certain DNA sequences appear to have characteristic methylation that is common among each cell sub-type, so that the DNA sequences can be arranged in families of differentially methylated regions (DMRs) as shown to the left of the heat maps.

FIG. 2A is a heat map of data obtained from isolated leukocyte subtypes, with eight lanes from left to right having cell samples as indicated across the bottom of the heat map as follows: mMDSC; monocytes; gMDSC; granulocytes; B cells; CD4T cells; CD8 T cells; and natural killer cells (NK). These results of application of the method to the entire data set illustrate the informative difference in DMRs.

FIG. 2B plots the prevalence of six of these cell subtypes in blood predicted on the ordinates for six of these subtypes as a function of percent observed on the abscissas. A linear relationship was observed for all six cell subtypes. These data validate application of the method to estimate the numbers of cell subtypes in blood.

FIG. 3 is a graphical representation of an estimate of cell numbers of data obtained from methylation of the DMRs to determine gMDSC levels, in 72 glioma patients compared to controls of 656 normal subjects (Hannum et al. samples), comparing predicted percent on the ordinate with observed on the abscissa. The glioma patient samples contained significantly greater gMDSC levels than the normal subjects. The Wilcoxan rank-sum P=4.9E-15, i.e. 4.9×10⁻¹⁵.

FIG. 4A is a Kaplan Meier survival plot of two groups of glioma patients, those having a hazard ratio of about 1.00 (17 patients, upper curve) and those having a hazard ratio of greater than 1.00 (55 patients, lower curve). The median survival of the former group was 2,345 days, and that of the latter group 778 days. These data show that MDSC levels are useful for prognosis of outcome in glioma patients.

FIG. 4B is a table of hazard ratios of glioma patients characterized by age, gender, mutation (in a gene encoding isocitrate dehydrogenase, IDH, only; or in a gene encoding telomerase reverse transcriptase, TERT only), histology (glioblastoma, GBM, compared to non-GBM), and both mutation and histology. The estimated gMDSC values were compared with a large published control population using identical cell estimation methodologies. The results show a highly significant increase in gMDSC levels in glioma cases compared to control samples.

FIG. 5A is a graph comparing the distributions of mdNLR between glioma patients and a non-cancer comparison group.

FIG. 5B is a boxplot comparing mdNLR of glioma patients by tumor grade.

FIG. 5C is a boxplot comparing mdNLR of glioma patients by tumor molecular subtype.

FIG. 5D shows Kaplan-Meier survival curves stratified by mdNLR (<4 vs ≥4).

FIG. 5E shows Kaplan-Meier survival curves stratified by histopathology (GBM vs non-GBM) and mdNLR (<4 vs ≥4).

FIG. 5F is a boxplot and a table showing leukocyte cell subtype composition of whole blood calculated with the validated algorithm and optimized reference libraries using the IDOL procedure of Koestler D C et al. BMC Bioinformatics 17:120 (2016), published Mar. 6, 2016 and submitted as Appendix A in provisional application Ser. No. 62/413,380, and hereby incorporated herein by reference in its entirety.

FIG. 6 is a scatterplot graph displaying mean β-values of myeloid cells on the ordinate, and lymphoid cells on the abscissa, for identification of myeloid and lymphoid specific CpG probes. The scatterplot depicts Illumina 450K methylation β-values among isolated lymphocyte subtypes (X-axis: T cells, B cell, NK cells) and myeloid subtypes (Y-axis; granulocytes, monocytes). The lower right quadrant identifies loci which are unmethylated in myeloid cells and which are densely methylated in lymphocytes.

FIG. 7 is a scatterplot of the methylation derived neutrophil to lymphocyte ratio (NLR) as a function of β-values using probe cg00901982, showing correlation of myeloid locus with mdNLR. Data from this and from four other probes are shown in the inset.

FIG. 8 shows Cox proportional hazards model of MDSCs (using the small 27K platform) predicting survival in head and neck cancer. Hazard ratios were elevated in patients in stages II, III and IV, in those with oropharyngeal tumors, and in smokers, compared with stage I cancer or non-smoker control patients. The Cox proportional hazards model demonstrates that an increased NLR and increased gMDSC proportion have statistically significant, independent associations with worse prognosis in head and neck cancer when adjusting for potential confounders age, gender, smoking history, tumor site, and tumor stage.

FIG. 9 shows sequence identification numbers 1 to 100 with the Illumina cgXXXXXXXX identification and ProbeSeqA nucleotide sequences. Sequences of other portions of SEQ ID 1 through 100 cgXXXXXXXX CpG sites and other Illumina CpG sites are available in Koestler D C et al. BMC Bioinformatics 17:120 (2016) and at the Illumina website, respectively.

### DETAILED DESCRIPTION

Cellular lineage and somatic differentiation are regulated by epigenetic mechanisms including DNA methylation and accordingly, the pattern of methylation at phenotypically important CpG regions varies substantially across individual tissues, cell-types and specifically across the distinct leukocyte subtypes. See, Accomando W P et al. Genome Biol 15 (3): R50 (2014); Reinius L E et al. PLoS One 7: e41361 (2012); Khavari, D. A. et al. Cell Cycle Georget. Tex. 9 (19): 3880-3883 (2010); Houseman, E. A. et al. Curr Environ Health Rep 2 (2): 145-154 (2015); Houseman E A et al. BMC Bioinformatics 13:86 (2012); Koestler D C et al. BMC Bioinformatics 17:120 (2016). Many differentially methylated regions (DMRs) demarcate the different leukocyte subtypes, lineages and activation states. See, Michels K B et al. Nat Methods 10 (10): 949-955 (2013); Jaffe A E et al. Genome Biol 15 (2): R31 (2014); Reinius L E et al. PLoS ONE 7 (7): e41361 (2012); Houseman E A et al. Curr Environ Health Rep 2 (2): 145-154 (2015). Changes in DNA methylation at specific CpG sites in whole blood DNA methylation comparisons include the possibility that such changes arise from variation in the leukocyte composition between study samples. See, Jaffe A E et al. Genome Biol 15 (2): R31 (2014); Houseman E A et al. Curr Environ Health Rep 2 (2): 145-154 (2015). These changes in methylation patterns associated with varying cell proportions or by the state of activation of any type of leukocyte may confound EWAS analyses. See, Michels K B et al. Nat Methods 10 (10): 949-955 (2013); Jaffe A E et al. Genome Biol 15 (2): R31 (2014); Houseman E A et al. Curr Environ Health Rep 2 (2): 145-154 (2015); Houseman E A et al. BMC Bioinformatics 13:86 (2012).

Previously, a unique reference library has been established of the DNA methylation profile for different leukocyte subtypes in blood. See, Accomando W P et al. Genome Biol 15 (3): R50 (2014); Houseman E A et al. BMC Bioinformatics 13:86 (2012). This unique reference library can inform a selection algorithm to estimate the relative abundance of the distinct leukocyte subtypes in blood samples based on the algorithm choosing CpG DMRs that distinguish the leukocyte subtypes from one another. See, Reinius L E et al. PLoS One 7: e41361 (2012); Kulis M et al. Nat Genet 47 (7): 746-756 (2015); Lee S-T et al. Nucleic Acids Res 40 (22): 11339-11351 (2012); Wiencke J K et al. Epigenetics 11 (5): 363-368 (2016). The selection algorithm has been adopted to adjust EWAS data which confers the ability to discriminate DNA methylation differences reflecting changes in leukocyte sub-populations from other possibly environmentally induced or disease-associated methylation events. See, Accomando W P et al. Genome Biol 15 (3): R50 (2014); Houseman E A et al. BMC Bioinformatics 13:86 (2012). Methylation signatures of leukocyte subtypes can be used for specific cell-type proportion estimates, adjustment for potential confounding in whole blood derived methylation studies and to identify DNA methylation differences associated with pathology of specific disease states. See, Waite L L et al. Front Genet 7:23 (2016); Kim S et al. Epigenomics (8) 9:1185-1192 (2016).

Pathologically important leukocyte subtype DNA methylation signatures in whole blood samples have been shown to modulate in patients afflicted with specific diseases and contribute significantly to EWAS analysis. See, Kim S et al. Epigenomics (8) 9:1185-1192 (2016). DMRs among leukocyte subtypes explain variability in disease associations related to DNA methylation status of individual CpG sites in the leukocyte subtypes. See, Reinius L E et al. PLoS One 7: e41361 (2012); Kulis M et al. Nat Genet 47 (7): 746-756 (2015); Lee S-T et al. Nucleic Acids Res 40 (22): 11339-11351 (2012); Wiencke J K et al. Epigenetics 11 (5): 363-368 (2016).

Statistical methods leveraging the tissue-specificity of DNA methylation for deconvoluting the cellular mixture of heterogenous biospecimens, such as blood, offer a promising solution to more accurately deconvolute biospecimens. See, Hannum G et al. Mol. Cell 49 (2): 359-367 (2013); Liu Y et al. Nat Biotechnol 31 (2): 142-147 (2013); Ali O et al. Clin Epigenet 7 (1): 12 (2015). However, their performance depends entirely on the underlying library of methylation markers being used for deconvolution.

It is shown herein that optimized DMR libraries that explain differences in DNA methylation among leukocyte subtypes allow for identification of pathologically important leukocyte subtypes in biological samples obtained from patients afflicted with various disease states, including inflammatory diseases and cancer. Pathologically important leukocyte subtypes, such as myeloid derived suppressor cells (MDSCs), are analyzed to prognose and/or diagnose specific disease states in biological samples based on the methylation profiles exhibited by the leukocyte subtype in the sample.

Additional methods used herein and background information are found in research papers by Koestler D C et al. entitled “Improving cell mixture deconvolution by identifying optimal DNA methylation libraries (IDOL)”, published online Mar. 8, 2016, BMC Bioinformatics (2016) 17:120 and supplementary data, and by Kim S et al.entitled “Enlarged leukocyte referent libraries can explain additional variance in blood-based epigenome-wide associate studies”, published Aug. 16, 2016 Epigenomics, (2016 (8) 9, 1185-1192), and supplementary data. A portion of the invention herein was published in a paper by Wiencke J et al. entitled “Immunomethylomic approach to explore the blood neutrophil lymphocyte ratio (NLR) in glioma survival” Feb. 2, 2017 Clinical Epigenetics (2017) 9:10, and a paper by Koestler, D C et al. entitled “DNA methylation-derived neutrophil-to-lymphocyte ratio: an epigenetic tool to explore cancer inflammation and outcomes,” March 2017 Cancer Epidemiol Biomarkers Prev. (2017) 26 (3): 328-338. The contents of these papers are hereby incorporated herein by reference in their entireties.

The examples and the following claims are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references including issued patents, published patent applications and non-patent literature references cited in this application are hereby incorporated by reference herein in their entireties.

Differentially methylated regions (DMRs) within DNA isolated from whole blood can be used to estimate the proportions of circulating leukocyte subtypes. The term “immunomethylomics” is used herein to describe the application of these immune lineage DMRs to studying leukocyte profiles. This approach was here applied to peripheral blood DNA from 72 glioma patients with molecularly defined brain tumors, representing common patient groups with defined characteristic survival times and risk factors. The proportions of leukocyte subtypes in samples were estimated using deconvolution algorithms with reference DMR libraries from isolated leukocyte populations and Illumina 450K DNA methylation data. Then, the neutrophil to lymphocyte ratio (NLR) was calculated using methylation-derived cell composition estimates (mdNLR). The NLR is considered an indicator of immunosuppressive cells in cancer patients.

Examples herein show that elevated mdNLR scores were observed in glioma patients compared to mdNLR values of published controls. Significantly decreased survival times were associated with mdNLR ≥4.0 in Cox proportional hazards models adjusted for age, gender, tumor grade, and molecular subtype (HR 2.02, 95% CI, 1.11-3.69). Five myeloid-related CpGs were identified that were highly correlated with the mdNLR (adjusted R²≥0.80). Each of the five myeloid CpG loci was associated with survival when adjusted for the above covariates and offer a simplified approach for utilizing fresh or archived peripheral blood samples for interrogating a very small number of methylation markers to estimate myeloid immune influences in glioma survival. It is shown in examples herein that the mdNLR (based on DNA methylation) is a novel candidate methylation biomarker that represents immunosuppressive myeloid cells within the blood of glioma patients with potential application in clinical trials and future epidemiologic studies of glioma risk and survival.

Abbreviations used herein include: AGS: Adult Glioma Study; DMR: Differentially methylated region; GBM: Glioblastoma; HR: Hazard ratio; IDH: Isocitrate dehydrogenase; mdNLR: Methylation-derived neutrophil lymphocyte ratio; NLR: Neutrophil lymphocyte Ratio; TERT: Telomerase reverse transcriptase; TMZ: Temozolomide.

About 14,000 Americans are diagnosed each year with glioma, the most common primary malignant brain tumor. See Dolecek T A Neuro Oncol. 14 Suppl 5: v1-49; 2012. Traditional histopathological criteria, including age and certain tumor markers, are currently being used to assess glioma patient prognosis. See, Louis D N et al. Acta Neuropathol. 114:97-109 (2007). Glioblastoma (GBM) patients, classified by the World Health Organization (WHO) as grade IV glioma, have a dismal prognosis with an estimated median survival of only 14.6 months. Younger patients and those with isocitrate dehydrogenase (IDH) mutated tumors have more favorable survival.

The standard therapies for high-grade glioma, which include surgery, temozolomide (TMZ) chemotherapy, and radiation, have led to relatively modest improvements in survival. See, Stupp R et al. N Engl J Med. 352:987-96 (2005). Previously, three key molecular features of glioma were demonstrated, telomerase (TERT) promoter mutation, IDH mutation, and 1p/19q codeletion, as sufficient to create an integrated molecular classification that defines five principal groups of glioma with characteristic distributions of age at diagnosis, clinical behavior, acquired genetic alterations, and associated germline variants. See, Eckel-Passow J E et al. N Engl J Med. 372:2499-508 (2015). Among these groups, IDH mutant only and TERT mutant only tumors are the most common and comprise about 75% of adult glioma patients. See, Eckel-Passow J E et al. N Engl J Med. 372:2499-508 (2015).

While the molecular classification of tumors has substantially improved our understanding of glioma prognosis, immune factors are notably absent in existing prognostic models. This omission is significant as immune evasion is a recognized hallmark of cancer (Hanahan D. Cell. 144:646-74, 2011), and there is abundant evidence that glioma patients suffer systemic immune defects, with the most profound alterations occurring in GBM patients, Grossman S A et al. Clin Cancer Res. 17:5473-80 (2011); Parney I F Adv Exp Med Biol. 746:42-52 (2012); Rolle C E et al. Adv Exp Med Biol. 746:53-76 (2012); Waziri A. Neurosurg Clin N Am. 21:31-42 (2010); and Yovino S et al. Cancer Invest. 31:140-4 (2013). Recent studies have emphasized the important role of developmentally immature and aberrantly activated myeloid-derived cells as contributing to cancer immunosuppression and adversely affecting patient survival. See, Gabrilovich D I et al. Nat Rev Immunol. 9:162-74 (2009); Hagerling C et al. Trends Cell Biol. 25:214-20 (2015); and Parker K H et al. Adv Cancer Res. 128:95-139 (2015). Furthermore, immune interventions represent a potentially powerful new therapeutic approach in glioma. See, Binder D C et al. Oncoimmunology. 11; 5 (2) (2015) e1082027 (2016) and Lin Y et al. Expert Opin Biol Ther 10:1265-1275 (2016).

The peripheral blood neutrophil to lymphocyte ratio (NLR), which can be derived using the common five-part white blood cell differential (neutrophils, basophils, eosinophils, monocytes, lymphocytes), has emerged as a surprisingly robust marker of cancer associated inflammation. See, Guthrie G J et al. Crit Rev Oncol Hematol. 88:218-30 (2013). Increases in the blood NLR have been remarkably consistent in their association with poor cancer survival. A recent meta-analysis including 100 independent studies encompassing over 40,000 subjects demonstrated that an elevated NLR was a statistically significant predictor of poor overall survival, cancer-specific survival, as well as progression free and disease free survival, even after adjustment for established risk predictors. See, Templeton A J et al. J Natl Cancer Inst. 2014; 106 (6): dju124 (2014). There are four studies showing shorter survival times in glioma patients with an elevated NLR. See, Bambury R M, et al. J Neurooncol. 114:149-54 (2013); Alexiou G A et al. J Neurooncol. 115:521-2 (2013); and McNamara M G et al. J Neurooncol. 117:147-52 (2014). Importantly, however, no study has taken into account the molecular features of glioma in conjunction with the NLR or other immune factors.

A goal of examples herein was to apply a new epigenetic approach to immune profiling to explore myeloid-related blood markers in glioma survival. Specifically, we examined the peripheral blood DNA methylation status of glioma cases using bioinformatic algorithms that deconvolute the complex methylation signature of whole blood into its component cell compartments. See, Houseman E A et al. BMC Bioinf. 13:86 (2012); Houseman E A et al. BMC Bioinf. 16:95 (2015); Houseman E A et al. Curr Environ Health Rep. 2:145-54 (2015); and Koestler D C et al. Epigenetics. 8:816-26 (2013).

The term “algorithm” as used herein refers not to a pure mathematical abstraction, but to an algebraic expression which is a statistical tool to transform biological data for computation. The statistical tools are applied to data by software packages using components programmed with such software. This approach to immune studies is based on recent epigenetic discoveries showing that differentially methylated regions (DMRs) provide highly specific and quantitative markers of immune cell profiles. See, Accomando W P et al. Genome Biol. 5; 15 (3): R50 (2014) and Koestler D C et al. BMC Bioinf. 17:120 (2016). As shown herein, an algorithm was developed and validated to estimate the NLR from 450K (450,000 different CpG containing sequences) methylation data (methylation-derived NLR; mdNLR). See, Koestler D C et al. Cancer Epidemiol Biomarkers Prev. 26 (3): 328-338 (2017), doi: 10.1158/1055-9965.EPI-16-0461, incorporated herein by reference. Results showed strong agreement between mdNLR and cytological NLR, and elevated mdNLR that was significantly associated with diminished patient survival times in head and neck squamous cell carcinoma and bladder cancer, as well as breast and ovarian cancer risk (Koestler D C et al. Cancer Epidemiol Biomarkers Prev. 26 (3): 328-338 (2017), doi: 10.1158/1055-9965.EPI-16-0461), paralleling the relationship between cytological NLR and cancer survivorship. See, Templeton A J et al. J Natl Cancer Inst. 2014; 106 (6): dju124 (2014). Data herein show the association of the mdNLR with survival among glioma patients.

Because altered myeloid differentiation is implicated in immune alterations in glioma, also explored was the idea that associations of mdNLR in glioma may be linked to myeloid-specific developmental CpG loci. Myeloid versus lymphoid specific CpGs were identified on the 450K array that strongly correlate with the mdNLR. This provides important evidence that the NLR is a surrogate marker of myeloid suppression. Consequently, both the mdNLR and the myeloid single CpGs are potential markers of skewed myeloid profiles which are useful in characterizing immune defects associated with survival in glioma.

An additional embodiment of the invention provides an array for determining methylation status of leukocyte types in a biological sample by analyzing methylation of a plurality of CpG dinucleotides in a plurality of genes of the sample, the array having a surface having a plurality of oligonucleotide probes with nucleotide sequences selected from at least one of the group of SEQ ID NO: 1-100, each probe attached at an addressable location on the surface, each probe hybridizes to a nucleotide sequence of a methylated form or an unmethylated form of a CpG dinucleotide in a sequence of a gene in the sample. In an embodiment of the invention, the biological sample is subjected to sodium bisulfite conversion before the sample is subjected to methylation status analysis on the array. Sodium bisulfite conversion is a chemical modification that differentially affects methylated cytosine nucleotides compared to unmethylated cytosine nucleotides.

The array in various embodiments has at least 5 probes, at least 10 probes, at least 25 probes, at least 50 probes, at least 100 probes, or at least 500 probes. The array can contain additional oligonucleotide probes attached to the array containing CpG dinucleotides that optimally discriminate among leukocyte types according to methylation status of CpG dinucleotides in a gene of the leukocyte type, and/or contains control probes. In various embodiments the oligonucleotide probes of SEQ ID NO: 1-100 are selected to function to distinguish CpG methylation profile DNA sequences of at least two leukocyte types selected from the group of: myeloid-derived suppressor cells (MDSCs), granulocytic MDSCs (gMDSCs), mMDSCs, mast cells, basophils, neutrophils, eosinophils, monocytes, natural killer cells (NK), activated NK cells, NKT cells, Th17 T cells, megakaryocytes, erythrocytes, cytotoxic T cells, double positive T cells, T helper cells, Treg cells, and B cells.

An embodiment of the invention provides a method of using an array to determine proportions of leukocyte types and prognose and/or diagnose a disease state in a biological sample of a subject, the method having steps of:
analyzing extent of hybridization of patient sample DNA to each of a plurality of oligonucleotide probes, the probes being affixed to at least two surfaces for each of methylated and unmethylated CpG sequences and otherwise identical in nucleotide sequence, the plurality of the nucleotide sequences selected from at least one of the group of SEQ ID NO: 1-100, for determining methylation status of at least one CpG dinucleotide in the DNA of the sample;
comparing methylation status of the plurality of CpG dinucleotides analyzed in the patient sample to a DNA methylation reference library, to determine proportion of each leukocyte type in the sample;
displaying the methylation status of the plurality of hybridized genes in the sample in a graphical representation, thereby generating an image of the methylation profile (methylome) of the leukocyte types in the patient sample; and,
prognosing and/or diagnosing a disease state in the patient associated with the methylation status of CpG sites in leukocyte types, the disease state selected from a cancer, a cardiac condition, inflammation, an autoimmune disease, and infection/sepsis.

The method of prognosing and/or diagnosing further includes, in a particular embodiment associating the methylation status of CpG sites in specific leukocyte types being above a pre-determined statistical threshold by determining a multivariate proportional hazards ratio equal to or greater than 1.0 as an indicium of a prognosis of an increased risk of death in the patient from the disease or as a diagnosis of the disease.

For example, the prognosing and/or diagnosing may further include associating the proportions of specific leukocyte types above a pre-determined statistical threshold of a neutrophil to lymphocyte ratio (mdNLR) equal to or greater than 1.0, equal to 2.0, or equal to or greater than 4.0 as an indicium of a prognosis of an increased risk of death in the patient from the disease or as a diagnosis of the disease.

In other embodiments, the prognosing and/or diagnosing may further include associating myeloid derived suppressor cell (MDSC), or gMDSC proportions in the sample as greater than or equal to a pre-determined statistical threshold of a multivariate proportional hazard value equal to or greater than 1.0, greater than 2.0, or equal to or greater than 2.5 as an indicium of a prognosis of an increased risk of death in the patient from the disease or as a diagnosis of the disease.

An embodiment of the invention provides a composition for analyzing proportions of specific leukocyte types in a biological sample, the composition comprising at least one oligopeptide selected from the group of SEQ ID NO: 1-100, and the leukocyte types selected from at least one of: myeloid-derived suppressor cells (MDSCs), granulocytic MDSCs (gMDSCs), mMDSCs, mast cells, basophils, neutrophils, eosinophils, monocytes, natural killer cells (NK), megakaryocytes, erythrocytes, cytotoxic T cells, double positive T cells, T helper cells, Treg cells, and B cells.

In a method of predicting a methylation class membership of leukocytes in a bodily fluid sample of a patient, the methylation class membership corresponding to an epigenetic signature of a plurality of leukocyte types, in which the method includes steps of measuring amounts of DNA methylation in each of a plurality of leukocyte type populations to determine differentially methylated regions (DMRs), ranking leukocyte DMRs for each leukocyte type according to statistical strength of association of each of at least one DMR with each leukocyte type, clustering samples in a training set using a defined number of highest ranked leukocyte DMRs to determine clustering solutions, a clustering solution corresponding to the methylation class membership, and predicting the methylation class membership for the leukocyte types within a testing set by applying the clustering solutions obtained from the training set to highest ranked leukocyte DMRs in the testing set, the predicted methylation class membership being determined by testing association of the predicted methylation class membership with the statistical discriminatory strength of the at least one DMR among the leukocyte types, the invention in some embodiments provides an improvement which is:
identifying statistically predictive subset DNA methylation libraries by scanning candidate sets of putative leukocyte-specific methylation markers to find CpGs that characterize leukocyte types in the sample estimated by a cell mixture deconvolution;
using a selection algorithm iteratively to construct and evolve subset libraries of DMRs consisting of CpG sites differentially methylated among leukocyte types, by selecting subsets of DMRs at each iteration of the algorithm based on the statistical contribution of each DMR to methylation class membership prediction accuracy;
modifying a probability of selection of the DMRs by the selection algorithm at each iteration of the algorithm, the probability of selection of a CpG being modified proportional to contribution of the at least one DMR to methylation class membership prediction accuracy; and,
applying the subset library to prognosis and/or diagnosis of cancer in the sample from the patient, in comparison to a plurality of control samples from a plurality of normal patients.

The term “algorithm” as used herein refers not to a pure mathematical abstraction, but to an algebraic expression which is a statistical tool for calculations to be performed by a computer programmed with software containing the algorithm, to transform the biological data through the computation into data detailing percentages of subtypes of white blood cells in blood. The improvement in some embodiments further includes identifying by scanning CpGs to assemble the candidate set of the leukocyte type-specific DMRs statistically associated with the leukocyte type methylation class membership.

Alternatively, the improvement in other embodiments further includes identifying by determining a methylation signature for the sample as a statistical weighted mixture, to obtain statistical weights proportional to the leukocyte type composition of the sample.

In yet another embodiment, the identifying further includes identifying the statistically predictive subsets of DMRs from the candidate sets of putative DMRs by comparing R² and Root Mean Square Error (RMSE) values between observed sample composition measurements of the testing set and predicted leukocyte cell type proportions obtained from the training set of at least one known DMR library.

In some embodiments the improvement further includes the subset DNA methylation libraries having at least 50 CpG sites, at least 100 CpG sites, at least 500 CpG sites, at least 700 CpG sites, or at least 900 CpG sites.

Alternatively in other embodiments, the subset DNA methylation libraries include less than 1,000 CpG sites, less than 800 CpG sites, less than 500 CpG sites, less than 200 CpG sites, or less than 100 CpG sites.

In another embodiment, the improvement includes modifying of the probability of selection in iterating the selection algorithm at least thousand-fold thereby evolving the DMR selection probabilities at each iteration proportional to contribution of the DMR to methylation class membership prediction accuracy, thereby preferentially selecting statistically predictive subset DMR libraries.

In another embodiment, the identifying further includes analyzing samples for DNA methylation profiles using an array platform.

An embodiment of the invention provides a method of using a selection algorithm for selection probabilities of leukocyte DMRs for inclusion in a statistically predictive subset library of DMRs for predicting leukocyte type methylation class membership of leukocytes in a blood sample from a patient for prognosis and/or diagnosis of a cancer in the patient, the method having steps of:
constructing a candidate DMR search space to compare mean methylation values among leukocyte types by identifying candidate CpGs that uniquely characterize each leukocyte cell type;
randomly assembling subset DMR libraries through multiple algorithm iterations;
estimating leukocyte cell compositions of the sample using the assembled subset DMR libraries and cell mixture deconvolution;
assessing the accuracy of leukocyte cell composition estimates by comparing statistical differences among observed cell compositions obtained by at least one method selected from the group of: fluorescence-activated cell sorting (FACS) and complete blood cell counts (CBC), to predicted cell compositions from cell mixture deconvolution, and implementing an iterative leave-one out procedure to assess the individual contribution of each CpG to statistical prediction performance of methylation class membership, and updating subset DMR libraries selection probabilities by modifying the CpGs selected using statistical weight of a relative and an absolute prediction accuracy of each CpG compared to the remaining CpGs in the library; and,
using the updated probabilities in successive iterations, the resulting subset DNA methylation libraries being comprised of the CpGs with the largest selection probabilities that contribute most significantly to improved accuracy of predicting leukocyte type methylation class membership.

The method of constructing further includes in a particular embodiment fitting a series of two-sample t-tests (or similar methodology) to the (J) arrayed CpGs and using the fitting to compare mean methylation beta-values between each leukocyte cell type against the mean beta-values computed among the other leukocyte cell types,
identifying the L/2 CpGs with the largest t-statistics and the L/2 CpGs with the smallest t-statistics for each of the K cell types, where L is a tuning parameter representing the number of cell-specific DMRs,
constructing a set Q, which consists of the L cell-specific DMRs, wherein Q is comprised of P=L×K putative DMRs, and represents the candidate search space for the subsequent steps of the selection algorithm, wherein the L is selected to be arbitrarily large to ensure a broad enough candidate search space, the user further pre-selecting J*<<P, representing the library size.

The method of randomly assembling further includes in a particular embodiment randomly selecting at iteration [symbol] , J* CpGs from [symbol] with probability π( [symbol] )j, j=1, 2, . . . ,P and at iteration 0, each CpG among the P candidate DMRs has an equal chance of being selected, determined by the equation π(0)j=1/P, ∀ j∈Q, wherein [symbol] ⊂Q represents the randomly assembled DMR library, comprising the J* randomly selected CpGs at iteration [symbol] .

The method of estimating further includes in a particular embodiment using the randomly assembled library [symbol] , applying cell mixture deconvolution to a training set to obtain cell composition estimates: ω^{~}i, where i=1, . . . ,N₁ and N₁ represents the number of training samples,
the applying resulting in a set of predictions given as Ω^{~}=[ω^{~}1,ω^{~}2, . . . ,ω^{~}N1], where 0≤ω^{~}i≤1 is a K×1 vector of the predicted cell proportions for training sample I,
further defining Ω^{~}k=[ω^{~}1k,ω^{~}2k, . . . ,ω^{~}N1k] as the predicted proportions for cell type k across the N₁ training samples.

The method of assessing further includes in a particular embodiment assessing prediction performance where relative and absolute measures are implemented using both the R² and root mean square error (RMSE) as the basis for assessments, where

Ω=[ω₁, ω₂, . . . , ω_{N1}] represents observed cell proportions for the N₁ target samples obtained via CBC, FACS, etc., and the proportion of variation in observed fraction of cell-type k (⋅ₖ) explained by its predicted fraction (Ω^{~}k) is computed as:

$${R}_{k}^{2} = 1 - \frac{{1}_{{N}_{1}}^{′} {( {Ω}_{k} - {\hat{Ω}}_{k} )}^{′} ( {Ω}_{k} - {\hat{Ω}}_{k} ) {1}_{{N}_{1}}}{{1}_{{N}_{1}}^{′} {( {Ω}_{k} - {\hat{Ω}}_{k} )}^{′} ( {Ω}_{k} - {\hat{Ω}}_{k} ) {1}_{{N}_{1}}} , 0 \leq {R}_{k}^{2} \leq 1$$

wherein

$${\overline{Ω}}_{k} = {\sum }_{i = 1}^{{N}_{1}} {Ω}_{k} / {N}_{1}$$

is an estimate of the mean observed fraction of cell-type k and \hat{Ω}ₖ represents the linear predictor obtained from regressing Ωₖ on \hat{Ω}k, wherein \hat{Ω}ₖ=\hat{β}ₖ [symbol] where, \hat{β}ₖ=( [symbol] )⁻¹ [symbol] Ωₖ and thus

$${\overline{R}}^{2} = \frac{1}{K} {\sum }_{k = 1}^{K} {R}_{k}^{2}$$

represents an estimate of the mean coefficient of determination across the K cell types, and the RMSE for cell type k=1, 2, . . . , K is computed using the following expression:

$${RMSE}_{k} = \sqrt{\frac{{1}_{{N}_{1}}^{′} {( {Ω}_{k} - {\tilde{Ω}}_{k} )}^{′} ( {Ω}_{k} - {\tilde{Ω}}_{k} ) {1}_{{N}_{1}}}{{N}_{1}}} ,$$

$$0 \leq {RMSE}_{k} < ∞$$

with

$$\overline{M} = \frac{1}{K} {\sum }_{k = 1}^{K} {RMSE}_{k}$$

representing an estimate of the mean RMSE across the K cell types, wherein both M̅ and R̅² are used for determining the contribution of each CpG in [symbol] on overall prediction performance.

The method of implementing an iterative leave-one out procedure further includes in a particular embodiment iteratively removing each of the J* CpGs contained in [symbol] to obtain the following sets Q( [symbol] )−j, which include all CpGs in [symbol] , except for CpG j,
repeating the steps according to claims 20 and 21 for each reduced library and using

$${\text{?}}_{- j}^{( ℓ )}$$

to obtain

$$( {\overline{M}}_{- j} , {\overline{R}}_{- j}^{2} )$$

which are estimates of the overall RMSE and coefficient determination when CpG j is excluded from the DMR library, and in subsequent iterations of the selection algorithm CpGs whose

$$\overline{M} - {\overline{M}}_{- j} < 0 and {\overline{R}}^{2} - {\overline{R}}_{- j}^{2} > 0.$$

The method of updating subset DMR libraries selection probabilities by modifying CpG selection probabilities further includes in a particular embodiment normalizing both M̅₋ⱼ and

$${\overline{R}}_{- j}^{2}$$

to obtain U₋ⱼ and V₋ⱼ, j=1, . . . . J* respectively by the equations

$${U}_{- j} = \frac{{\overline{M}}_{- j} - \overline{M}}{sd ( {\overline{M}}_{- j} )} ,$$

$${V}_{- j} = \frac{{\overline{R}}_{- j}^{2} - {\overline{R}}^{2}}{sd ( {\overline{R}}_{- j}^{2} )}$$

where −∞<U₋ⱼ<∞ and −∞<V₋ⱼ<∞,
generating a composite measure of probability of selection by first converting (U₋ⱼ, −V₋ⱼ) from the Cartesian coordinate system to the polar coordinate system using the equations

$${r}_{- j} = \sqrt{δ {U}_{- j}^{2} + ( 1 - δ ) {( - {V}_{- j} )}^{2}}$$

$${θ}_{- j} = atan 2 ( - ( 1 - δ ) {V}_{- j} , δ {U}_{- j} )$$

where atan2 is a common variation of the arc tangent function, r₋ⱼ is the radial coordinate, θ₋ⱼ is the angular coordinate, and 0≤δ≤0 is a parameter that controls the balance between relative and absolute prediction performance,
modifying the selection probability of CpG j by the increment

$${p}_{- j} = {r}_{- j} cos ( {θ}_{- j} - π / 4 ) , - ∞ \leq {p}_{- j} \leq ∞ ,$$

updating selection probabilities by equation (3)

$${π}_{j}^{( ℓ + 1 )} = \frac{{ρ}_{j}^{( ℓ + 1 )}}{{\sum }_{j ∈ \text{?}} {ρ}_{j}^{( ℓ + 1 )}} , 0 \leq {π}_{j}^{( ℓ + 1 )} \leq 1 \qquad ( 3 )$$

wherein

$${ρ}_{j}^{( ℓ + 1 )} = \{ \begin{matrix}{π}_{j}^{( ℓ )} exp it ( {p}_{- j} ) + {π}_{j}^{( ℓ )} / 2 & if j ∈ \text{?} \\ {π}_{j}^{( ℓ )} & if j ∉ \text{?}\end{matrix} \qquad ( 4 )$$

and expit is the inverse-logit function, i.e., expit(x)=exp(x)/(1+exp(x)), thereby selection probabilities for each j∈ [symbol] are modified based on how beneficial/not beneficial each CpG was determined to be in the presence of the remaining J*−1 CpGs, the probability of selection being unchanged for CpGs j∉ [symbol] as well as for CpGs where p₋ⱼ≈0.

The method of updating selection probabilities by modifying CpG selection probabilities further includes in a particular embodiment using the updated probabilities, π( [symbol] +1)j, j=1, . . . ,P, in repeating the steps according to claims 20-24 for thousand-fold iterations, the final solution consisting of the subset DMR library comprised of the J* CpGs with the largest selection probabilities.

The method of updating further includes in some embodiments when δ=1/2, a CpG's influence on relative and absolute prediction performance receives equal weight, when δ→1 a CpG's influence on absolute prediction performance receives more weight; and, when δ→0, a CpG's influence on relative prediction performance receives more weight.

The method of updating further includes in some embodiments when δ=1/2, CpGs with the largest increment in selection probability (i.e., large p₋ⱼ) are those with large r₋ⱼ and θ₋ⱼ close to π/4 radians, CpGs with the largest decrease in selection probability (i.e., small p₋ⱼ) are those with large r₋ⱼ and θ₋ⱼ close to 5π/4, and when p₋ⱼ≈0, this implies that either r₋ⱼ is small or θ₋ⱼ is close to (3π/4, −π/4) radians and suggests that withholding CpG j from ⁽ [symbol] ⁾ is neither helpful nor detrimental to prediction performance.

The method of updating further includes in some embodiments determining J* by fitting the selection algorithm across a range of possible values for J*, (i.e., J*={50,100,200, . . . }) followed by comparing prediction performance across each of the specified values, selecting the smallest value of J* upon which the gains in prediction performance for increasing values of J* is minimal, (i.e., within some predetermined tolerance of the performance metrics).

The method of the improvement of the statistically predictive subset DNA methylation libraries further includes in some embodiments CpGs whose methylation signature is maximally distinct among the leukocyte cell types and whose methylation signature variation is minimal within a given leukocyte cell type.

In a method of predicting a methylation class membership of leukocytes in a bodily fluid sample of a patient, the methylation class membership corresponding to an epigenetic signature of a plurality of leukocyte types, in which the method includes steps of measuring amounts of DNA methylation in each of a plurality of leukocyte type populations to determine differentially methylated regions (DMRs), ranking leukocyte DMRs for each leukocyte type according to statistical strength of association of each of at least one DMR with each leukocyte type, clustering samples in a training set using a defined number of highest ranked leukocyte DMRs to determine clustering solutions, a clustering solution corresponding to the methylation class membership, and predicting the methylation class membership for the leukocyte types within a testing set by applying the clustering solutions obtained from the training set to highest ranked leukocyte DMRs in the testing set, the predicted methylation class membership being determined by testing association of the predicted methylation class membership with the statistical discriminatory strength of the at least one DMR among the leukocyte types, the invention provides an improvement in some embodiments which is
obtaining leukocyte methylation data of the sample using an array containing a plurality of nucleotide sequences each having a CpG site affixed to the array;
identifying statistically predictive subset DNA methylation libraries by scanning candidate sets of putative leukocyte-specific methylation markers to find sets of CpG sites that characterize each of the respective leukocyte types in the sample estimated by a cell mixture deconvolution;
using a selection algorithm iteratively to construct and evolve subset libraries of DMRs consisting of CpG sites differentially methylated among leukocyte types, by selecting subsets of DMRs at each iteration of the algorithm based on the statistical contribution of each DMR to methylation class membership prediction accuracy;
modifying a probability of selection of the DMRs by the selection algorithm at each iteration of the algorithm, the probability of selection of a CpG being modified proportional to contribution of the at least one DMR to methylation class membership prediction accuracy; and,
comparing the subset library of the patient DMRs sample to DMRs of a reference-based library of a plurality of control samples from a plurality of normal patients, to obtain a prognosis and/or a diagnosis of a cancer of the patient.

The improvement in some embodiments further includes applying the subset library by calculating a multivariate proportional hazards ratio for the sample from the patient to assess the relationship of cancer prognosis and/or diagnosis with methylation status of the leukocyte composition.

The improvement in some embodiments further includes obtaining the prognosis and/or diagnosis of cancer by selecting the leukocyte composition methylation status from the group of myeloid-derived suppressor cell (MDSC) methylation status and granulocytic myeloid-derived suppressor cell (gMDSC) methylation status. In some embodiments, calculating the gMDSC multivariate proportional hazards ratio equal to or greater than 1.0 is an indicium of a prognosis of an increased risk of death in the patient from the disease or is a diagnosis of the disease. For example, some embodiments further include associating the hazard ratio of about 1.0, or about 2.0 as an indicium of about a two-fold increase in the risk of death in the patient from the cancer. Some embodiments further include adjusting the multivariate proportional hazards ratio for tumor histology status, gene mutation status, patient age, patient history, and patient gender status.

The improvement in some embodiments may further include selecting the CpG sites for inclusion in the statistically predictive subset library those CpG methylation patterns that indicate MDSCs or gMDSCs in the sample.

An embodiment of the invention provides a method of using a selection algorithm for selection probabilities of leukocyte differentially methylated regions (DMRs) for inclusion in a statistically predictive subset library of DMRs for predicting leukocyte type methylation class membership of leukocytes in a blood sample from a patient for prognosis and/or diagnosis of a cancer in the patient, the method having steps of:
constructing a candidate DMR search space to compare mean methylation values among leukocyte types by identifying CpGs that uniquely characterize each leukocyte cell type, and randomly assembling subset DMR libraries with CpGs that uniquely characterize the leukocyte cell types through multiple algorithm iterations;
estimating leukocyte cell compositions in the sample using the assembled subset DMR libraries and cell mixture deconvolution, and computing leukocyte ratios from the estimated leukocyte compositions of the sample;
assessing the accuracy of leukocyte cell composition estimates by comparing statistical differences among observed cell compositions obtained by at least one method selected from the group of: fluorescence-activated cell sorting (FACS) and complete blood cell counts (CBC), to predicted cell compositions obtained from cell mixture deconvolution of normal control samples, and implementing an iterative leave-one out procedure to assess individual contributions of each CpG to statistical prediction performance of the methylation class membership of the leukocytes, and further computing a dispersion separability criterion (DSC) score to assess a DMR subset power for discriminating among leukocyte types, to select CpGs, and updating subset DMR library selection probabilities by modifying the CpGs selected using the statistical prediction performance of a relative and of an absolute prediction accuracy of each CpG compared to remaining CpGs in the library, and using the updated probabilities in successive iterations to obtain updated probabilities, resulting statistically predictive subset DNA methylation libraries containing CpGs with the largest selection probabilities for improved accuracy of predicting leukocyte type methylation class membership; and,
fitting the multivariate proportional hazards ratio calculated from the sample to the updated subset DMR libraries thereby prognosing and/or diagnosing cancer in the blood sample from the patient.

The method of computing leukocyte ratios from the estimated leukocyte cell compositions further includes in a particular embodiment comparing amounts of at least two different leukocyte types present in the leukocyte cell composition of the sample from the patient. The method of fitting the multivariate proportional hazards ratio further includes, in a particular embodiment comparing the hazard ratio to a Kaplan Meier plot of cancer survival data to prognose patient survival probability. The method of computing leukocyte ratios further includes in some embodiments calculating a neutrophil to lymphocyte ratio (mdNLR) and fitting the multivariate proportional hazards ratio to the mdNLR.

The method of updated statistically predictive subset DMR library further includes in some embodiments CpG sites of granulocytic myeloid-derived suppressor cells (gMDSCs) in the sample from the patient. The method of the statistically predictive subset DMR libraries further includes in some embodiments CpG sites whose methylation status indicates MDSCs in the sample from the patient.

The method of the dispersion separability criterion (DSC) score further includes in some embodiments defining the DSC as Db/Dw, wherein Db is a measure of dispersion between cell types and Dw is a measure of dispersion within cell types, and is implemented to quantify dispersion between leukocyte types and within leukocyte types for a randomly selected DMR subset.

### Examples

### Example 1. Patient Samples

Patients were chosen from the University of California San Francisco (UCSF) Adult Glioma Study (AGS) who had both archival blood and tumor marker data. See, Wrensch M et al. Neuro Oncol. 8:12-26 (2006). AGS participants represent primary glioma patients; no recurrent or secondary GBM cases were included. Seventy-two cases were selected from the two most prevalent molecular subtypes of glioma (Eckel-Passow J E et al. N Engl J Med. 372:2499-508, 2015) (i.e., cases with IDH mutation only or TERT promoter mutation only). Samples from cases aged 40 to 59 were selected as follows: all available non-GBMs and IDH-only GBMs were included. TERT-only GBMs were chosen to match the ages of both the IDH-only GBMs and the TERT-only non-GBMs. Blood samples were collected from patients a median of 100 days after they were histologically diagnosed. Clinical information was collected on patient treatments including temozolomide (TMZ) chemotherapy, radiation therapy, extent of surgery, and steroid use at the time of blood sampling. The anticoagulated whole blood was processed, and DNA was isolated and bisulfite converted as previously described (27).

### Example 2. Quality Control and Preprocessing of the DNA Methylation Data

Illumina 450K arrays were run by the UCSF Human Genomics core. Preprocessing and quality control was accomplished using the minfi Bioconductor package. See, Aryee M J et al. Bioinformatics. 30:1363-9 (2014). To ensure high-quality methylation data, CpG loci having a sizable fraction (>25%) of detection p values above a predetermined threshold (detection P>10E-5, i.e. 10⁵) were excluded. See, Wilhelm-Benartzi C S et al. Br J Cancer. 109:1394-402 (2013). Subset Quantile Within Array (SWAN) normalization was performed for type 1/2 probe adjustment, See, Maksimovic J et al. Genome Biol. 13 (6): R44 (2012). The presence of technical sources of variability induced by plate and/or BeadChip was examined using principal components analysis (PCA), and the top K principal components (Teschendorff A E et al. Bioinformatics. 27:1496-505, 2011) were examined in terms of their association with plate and BeadChip. If plate and/or BeadChip was found to be significantly associated with any of the top K principal components, we applied ComBat method (Johnson W E et al. Biostatistics. 8:118-27, 2007) for normalization using the sva Bioconductor package. The commercially available 450K library uses 450,000 CpG sites from the human genome, each name cgXXXXXXXX (eight numbers). The nucleotide sequences are available to users of the product, Illumina Human Methylated Bead Arrays, with the chromosomal location and associated probe sequence, as support that is downloaded for kits of arrays and beads.

### Example 3. Cell Mixture Deconvolution Analysis

Using the preprocessed and normalized methylation data, an optimized reference-based cell mixture deconvolution methodology (Koestler D C et al. BMC Bioinf. 17:120, 2016) was applied to gain insight into the cellular composition of the samples considered here. Specifically, the proportions of CD4+ T cells, CD8+ T cells, B cells, natural killer (NK) cells, monocytes, and granulocytes were estimated for each sample using the function “estimateCellCounts” in the minfi Bioconductor package using an optimized reference library set of CpGs.

Example 4. Computing the Methylation-Derived Neutrophil Lymphocyte Ratio (mdNLR)

Estimation of the mdNLR was carried out as previously described. See, Koestler D C et al. Cancer Epidemiol Biomarkers Prev. 26(3): 328-338 (2017), doi: 10.1158/1055-9965.EPI-16-0461. Briefly, the method requires three main steps: (i) identify differentially methylated CpGs among leukocyte subtypes, (L-DMRs), (ii) perform cell mixture deconvolution to estimate the proportion of leukocyte subtypes using L-DMRs identified in step 1, and (iii) compute the ratio of the predicted proportion of neutrophil granulocytes to lymphocytes. The mdNLR was computed by taking the ratio of predicted granulocyte and lymphocyte fractions, mdNLR i=ω \hat{ }(Gran,i)ω \hat{ }(Lymph,i), =ω\hat{ }(Gran,i)ω\hat{ }(Lymph,i), 0≤mdNLR i<∞. The mdNLR scores are based on beta values using 300 L-DMR CpGs. See, Koestler D C et al. BMC Bioinf. 17:120 (2016). A publicly available implementation of this method is available in the IDOL R package (https://www.r-project.org/). The IDOL R-package has been submitted to the Comprehensive R Archive Network (CRAN) and is available through Github

Example 5. Statistical Analyses of the mdNLR and Clinical Outcomes

Associations between mdNLR and clinical covariates were assessed using either logistic regression or linear regression models. Cox proportional hazards regression models were used to examine the association between mdNLR and survival time and were fit using the “coxph” function in the survival R package. Survival models were adjusted for established risk predictors and potential confounders, including age, gender, histological subtype (GBM versus non-GBM), and IDH/TERT mutation status (IDH-only mutation versus TERT-only mutation). The proportionality assumption was assessed by plotting the scaled Schoenfeld residuals against time, and the “cox.zph” function in the survival R package was used for testing the proportionality of each predictor included in the survival models herein. See, Grambsch P M et al. Biometrika 81:515-26 (1994). In the survival analyses, mdNLR was modeled both as a continuous predictor and by dichotomizing subjects into high and low mdNLR groups. The binary cut point of mdNLR>4 is based on previous studies. See, Bambury R M, et al. J Neurooncol. 114:149-54 (2013). The performance of different survival models that included known risk factors was compared with analyses including mdNLR and single locus CpGs. Three metrics were computed using the packages survival and survAUC to compare the performance of these models: concordance index (c-index), the Gerds and Schumacher Brier score, and the Song and Zhou (Gerds T A et al. Biom J. 48:1029-40, 2006) time-dependent area under the receiver operator characteristic curve (tAUROC) (Song X et al. Stat Sin. 18:947-65, 2008. Log-rank tests were used to judge differences between the experimental and baseline model. The baseline model contained patient age, gender, tumor grade, and mutation status (TERT mutant only vs IDH mutant only).

Example 6. Identification of Myeloid-Specific Single Locus Markers of the mdNLR

While the mdNLR requires 300 CpGs to estimate the neutrophil lymphocyte ratio (Koestler D C et al. Cancer Epidemiol Biomarkers Prev. 26 (3): 328-338, 2017), it was envisioned herein that the NLR (and the mdNLR) is a biomarker of the known influx of myeloid-derived suppressor cells into the peripheral blood that occurs with the development of a new cancer (Gabrilovich D I et al. Nat Rev Immunol. 9:162-74, 2009), and as a result of this, reasoned that there may exist individual influential CpGs arising during myeloid differentiation that could serve as surrogates for the mdNLR. To test this method, myeloid-specific markers were sought. The M values of 54 samples from the Reinius dataset (excluding the six whole blood samples, GSE35069 (Reinius L E et al. PLoS One 7: e41361, 2012) were modeled according to if they were predominantly myeloid or lymphoid cells, adjusting for the proportion of the blood cells in the samples as measured by flow cytometry. The top 100 loci were selected using the RnBeads automatic rank cutoff approach. A second model then evaluated the relationship between the mdNLR as the outcome and the top 100 myeloid-specific loci to obtain a reduced list of methylation-derived mdNLR surrogates. For variance stabilization, beta values were converted to M values and were modeled assuming linear, quadratic, and cubic relationships with survival time; adjusted R 2 values were then computed to assess the correlation of each methylation site. First the 100 myeloid-specific loci were modeled using the methylation data from subjects in this study and then the models were repeated in the Hannum (Hannum G et al. Mol Cell. 49:359-67, 2013) [GSE40279] and Liu (Liu Y et al. Nat Biotechnol. 31:142-7, 2013) [GSE42861] blood methylation datasets. For the top 10 models, the adjusted R² ranged between 40-86%. Five loci were consistently found to obtain an adjusted R2 greater than 80% in all three datasets. Each of the five loci was markedly demethylated in myeloid compared to lymphoid cells and stem cells (using ENCODE resources).

Example 7.Degree of Methylation in Isolated MDSCs Differs from that of Isolated Granulocytes

In order to assess differentially methylated regions a novel bioinformatic methodology called DMRSubsetFinder was used. The accuracy of cell composition estimates obtained through CMD is driven entirely by the underlying DMR library being used for deconvolution (Accomando W P et al. Quantitative reconstruction of leukocyte subsets using DNA methylation. Genome Biol, 2014. 15 (3): p. R50.: Koestler D C et al. Blood-based profiles of DNA methylation predict the underlying distribution of cell types: a validation analysis. Epigenetics, 2013.8(8): p. 816-26).

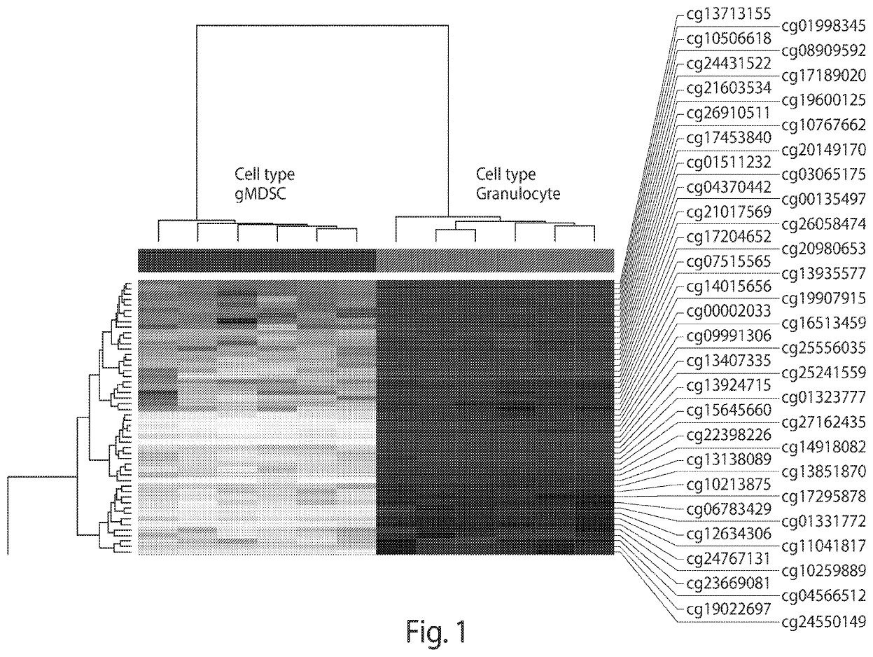

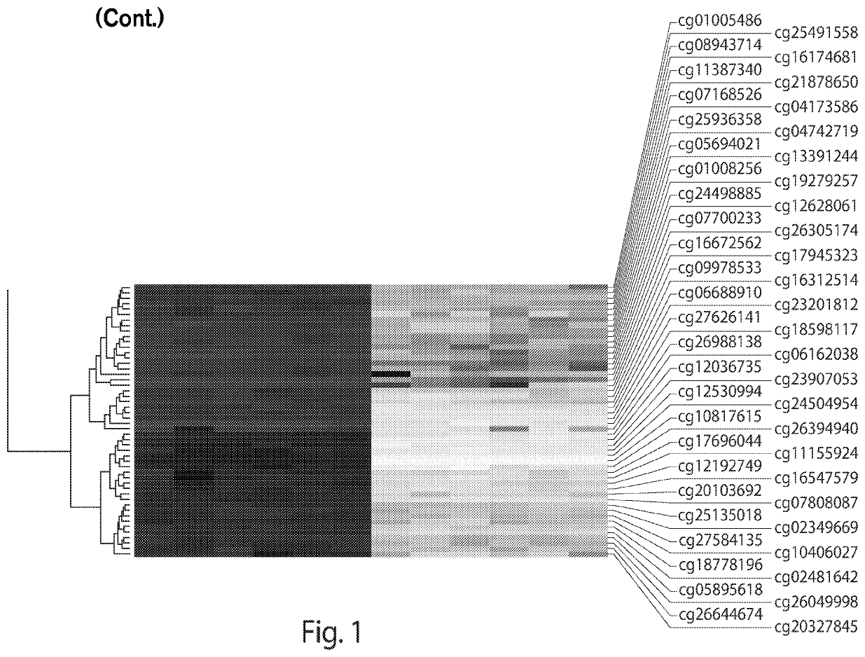

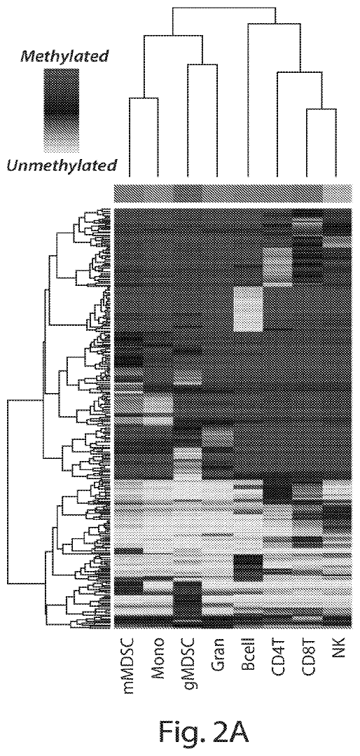

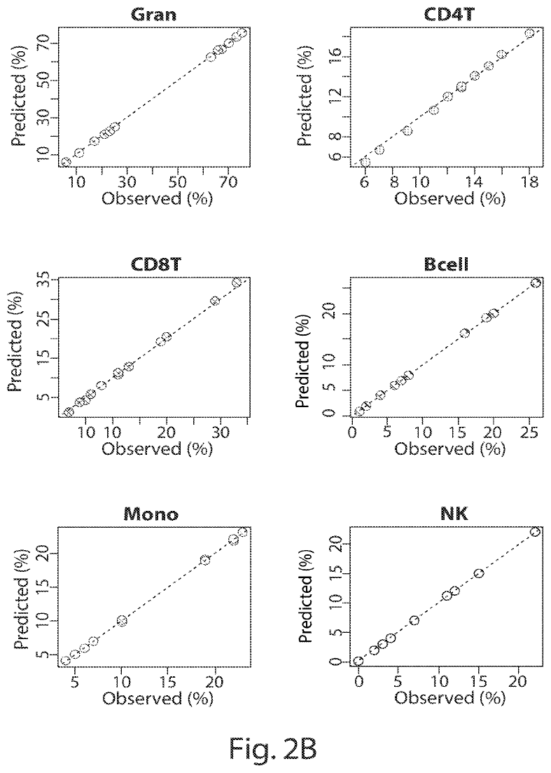

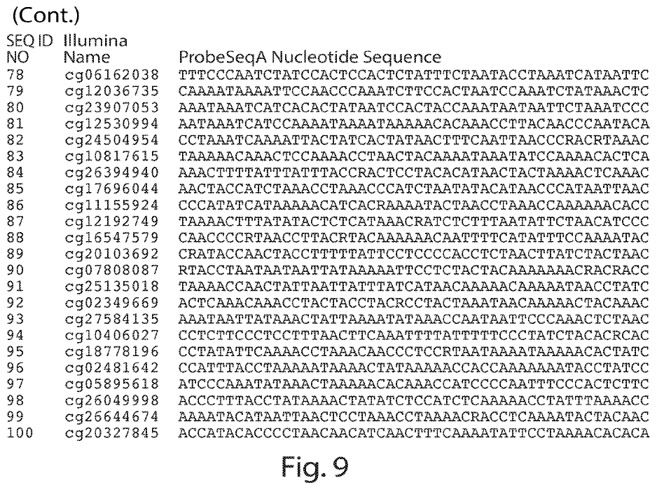

The DMRSubsetFinder, an iterative selection algorithm for identifying DMR libraries that provide optimal discrimination of the entire immune cell landscape, is a method developed in examples herein. FIG. 1 illustrates the numerous differences in methylation in myeloid-derived suppressor cells (MDSCs) compared with normal granulocytes. The CpG sites use are listed on the right hand side of the heatmap and the ProbeSeqA nucleotide sequences are shown in FIG. 9. FIG. 2A shows the results of application of the method to the entire data set, illustrating that informative differences in DMRs exist. FIG. 2B shows the validation of the application of the method in estimating the cell numbers. Applying estimates of cell numbers is shown in FIG. 3, revealing that MDSC numbers are significantly greater in newly diagnosed glioma patients.

Example 8. Application of gMDSC Assay to Glioma Patients: Comparison of gMDSC in Glioma Patients with a Normal Control Group and Predictive Value in Glioma Survival.

A pilot experiment was performed involving 450K methylation array analysis applied to blood from 72 UCSF AGS glioma patients. Patients were selected as IDH mutated only, as TERT promoter mutated only, or as grade II/III and IV patients with similar age and treatment status. The reference-based cell mixture deconvolution methodology, specifically, the proportions of CD4+ T-cells, CD8+ T-cells, B cells, natural killer (NK) cells, monocytes, granulocytes and gMDSC were estimated for each sample using the function “EstimateCellCounts” in the Bioconductor package minfi. In addition, the estimated cell fractions enabled computation of various WBC ratios, including, neutrophil to lymphocyte ratio (mdNLR). The unique CpG signature of the gMDSC isolated from neonatal cord blood was identified through the optimization algorithm as published (Koestler D C et al. BMC Bioinf. 17:120 (2016). The prevalence of gMDSC as well as other immune parameters was then estimated.

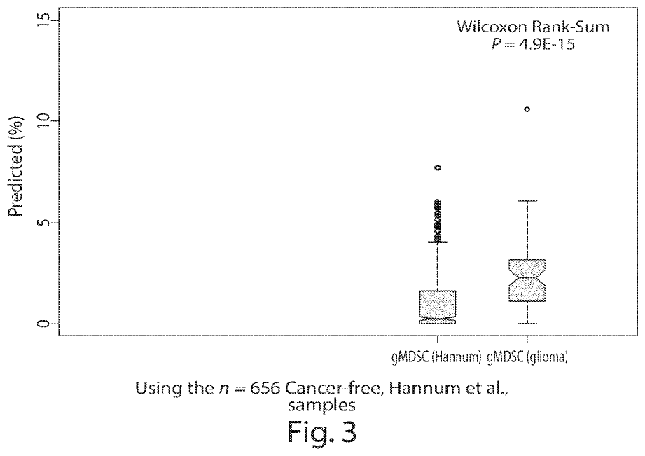

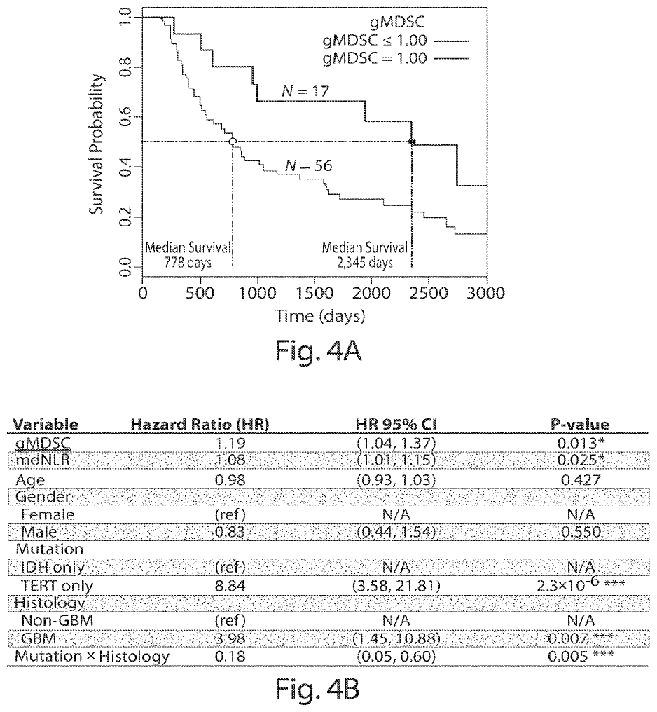

The estimated levels of gMDSC were compared with a large published control population using identical cell estimation methodologies as the glioma cases. The comparison in FIG. 4 shows a highly significant increase in gMDSC levels in glioma cases compared to controls. Importantly, earlier studies assessed the relationship of gMDSC with glioma survival.

Example 9. Relationship Between Estimated Blood Cell Composition Including gMDSC and Survival in Glioma (FIG. 4B)

Follow-up times for the 72 UCSF AGS glioma patients studied ranged from 162 days to 16 years post-diagnosis with about 80% of the participants having died during the follow-up period. Median survival was about 2.75 years post-diagnosis. Multivariate Cox proportional hazards (Cox-PH) models assessed the relationship of survival with WBC composition adjusted for tumor histology (GBM versus non-GBM), mutation status (IDH only versus TERT promoter interaction between histology and mutation status. Cox-PH models fit to proportion values for mdNLR and gMDSC glioma survival. In particular, elevated gMDSC (>1.0) was associated with a 2.0-fold increased hazard of death (p=0.02), consistent with a growing literature suggesting the clinical and prognostic value of gMDSC in some cancers although this is the first demonstration of significant survival advantage in human glioma.

### Example 10. Neutrophil Lymphocyte Ratio in Glioma Patients Assessed by Immunomethylomics

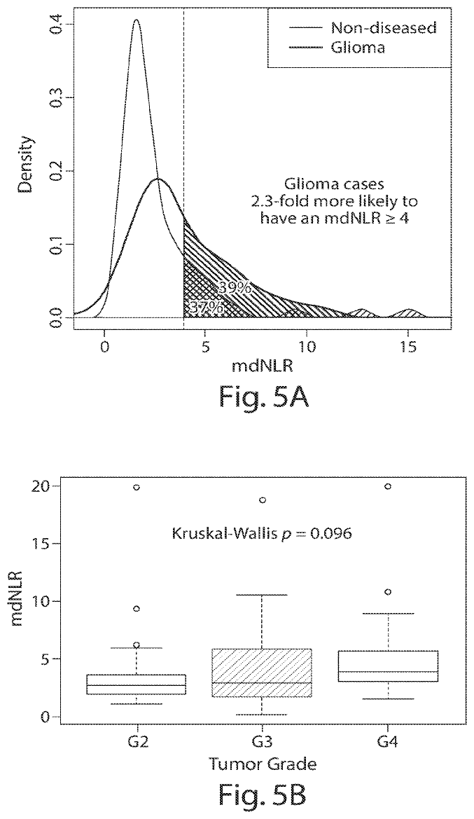

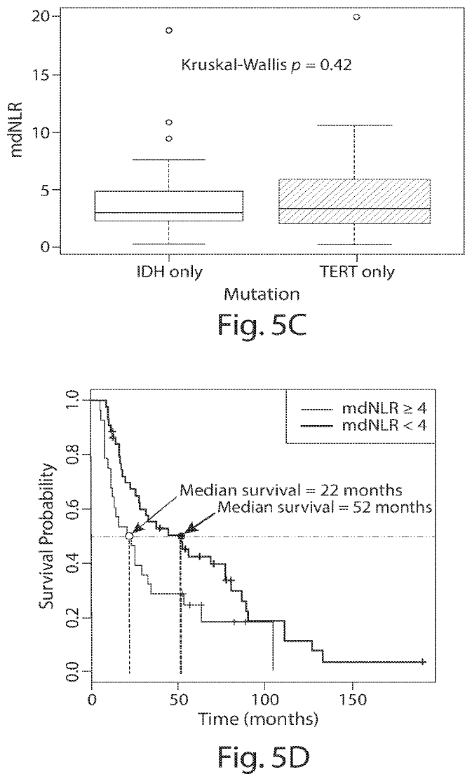

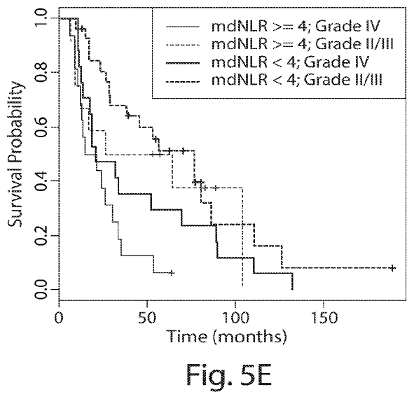

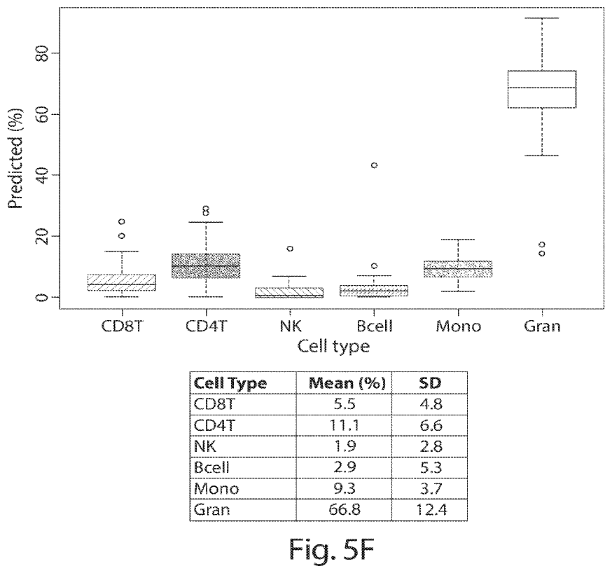

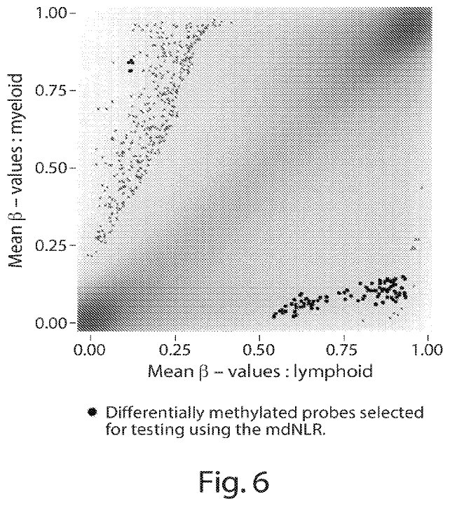

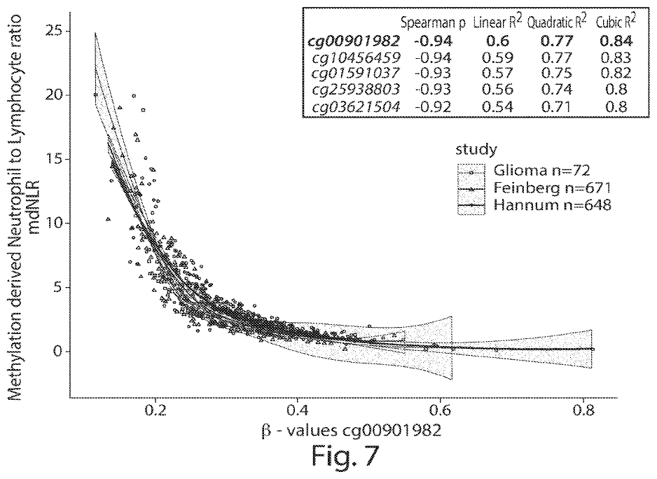

The study sample sizes, clinical characteristics, and available demographic/epidemiological data are given in Table 1. Leukocyte cell composition of whole blood was calculated with the validated algorithm and optimized reference libraries using the IDOL procedure (Koestler D C et al. BMC Bioinf. 17:120, 2016), FIG. 5G. Combining the myeloid and lymphocytic subtypes allowed the calculation of the mdNLR. The mdNLR scores among glioma cases were then compared with a large public database of blood methylation data collected on 656 non-cancer adults. See, Hannum G et al. Mol Cell. 49:359-67 (2013).

FIG. 5A compares the distributions of mdNLR among glioma cases and the non-cancer comparison group. It was observed that the median mdNLR of glioma patients was elevated compared to the non-cancer group. Higher glioma tumor grade was associated with increased mdNLR values (FIG. 5B). Further, mdNLR scores were similar among cases whose tumors contained IDH1 compared to cases whose tumors contained TERT promoter mutation (FIG. 5C).

TABLE 1
Summary of patient characteristics

| | |
|---|---|
| Number | 72 |
| Median age (IQR) | 47 (44, 54) years |
| Sex | |
| Male | 72% |
| Female | 28% |
| Histology and grade | |
| Astro/oligo/oligoastro gr II-III | 54% |
| Glioblastoma multiforme gr IV | 46% |
| Mutation status | |
| TERT promoter only | 58% |
| IDH-only | 42% |
| Methylation-derived NLR (mdNLR) | |
| mdNLR < 4 (%) | 61% |
| mdNLR ≥ 4 (%) | 39% |
| Length of follow-up | 5-190 months |
| Median survival time (IQR) | 29 (13, 65) months |

Example 11. Association of mdNLR with Glioma Survival Times

Median survival in cases in which it was observed that mdNLR<4 was 52 months compared to those with elevated mdNLR scores; 22 months (FIG. 5D). Kaplan-Meier survival curves were further stratified by histopathology (GBM vs non-GBM) and shorter survival times were observed among GBM cases, (FIG. 5E). Cox proportional hazards models that included known prognostic factors (age, grade, mutation status) indicated significant association of a high mdNLR (>4) with an increased risk of death; HR 2.02, 95% CI, 1.11-3.69, P=0.02 (Table 2). A Cox model including chemotherapy and steroid use indicates that mdNLR is associated with survival time, independent of therapy; HR 1.84, 95% CI, 1.00-3.38, P=0.049 (FIG. 4C).

Glioma grading was based on WHO 2007 criteria; however, since IDH mutation and 1p19q codeletion status was known, these cases were reclassified using the new WHO 2016 brain tumor classification. See, Louis D N et al. Acta Neuropathol. 131(6): 803-20 (2016). doi: 10.1007/s00401-016-1545-1. Based on the WHO 2016 criteria, two anaplastic oligodendroglioma or oligoastrocytomas cases would have been classified as GBM instead of non-GBM due to having evidence of microvascular proliferation. This reclassification would not have substantially altered the results of this analysis.

TABLE 5
Cox proportional hazards survival models including
mdNLR, age, grade, and tumor mutation status

| | | | | Univariate models | | Multivariate model | |
|---|---|---|---|---|---|---|---|
| | Number | Survival years | | HR | | HR | |
| | (deceased) | Mean | Median | (95% Cl) | p value | (95% Cl) | p value |
| mdNLR < 4 | 44(35) | 4.8 | 4.3 | Referent | | Referent | |
| | | | | group | | group | |
| mdNLR ≥ 4 | 28(23) | 3.1 | 1.8 | 1.78(1.03-3.07) | 0.038 | 2.02(1.11-3.69) | 0.022 |
| GBM | 33(32) | 2.9 | 1.7 | Referent | | Referent | |
| | | | | group | | group | |
| non-GBM | 39(26) | 5.3 | 5.3 | 0.48(0.28-0.81) | 0.006 | 1.06(0.56-2.00) | 0.859 |
| IDH only | 30(18) | 6.6 | 7.2 | | | Referent | |
| | | | | | | group | |
| TERT only | 42(40) | 2.4 | 1.4 | 4.19(2.34-7.48) | <0.0001 | 4.83(2.35-9.93) | 0.00002 |
| Age (continuous) | | | | | | 0.97(0.92-1.02) | 0.274 |
| No Chemo | 56(42) | 4.8 | 4.3 | | | Referent | |
| and/or | | | | | | group | |
| No Dex | | | | | | | |
| Chemo and | 16(16) | 2.0 | 1.3 | | | 1.86(0.99-3.51) | 0.055 |
| Dex | | | | | | | |

All covariates modeled met proportionality assumptions
mdNLR methylation-derived NLR (neutrophil to lymphocyte ratio)
HR = hazard ratio;
CI = confidence interval;
Chemo = last given chemotherapy within 90 days before blood draw;
Dex = taking dexamethasone at the time of the blood draw

Example 12. Association of Single CpG Myeloid Differentiation Loci with mdNLR and Survival

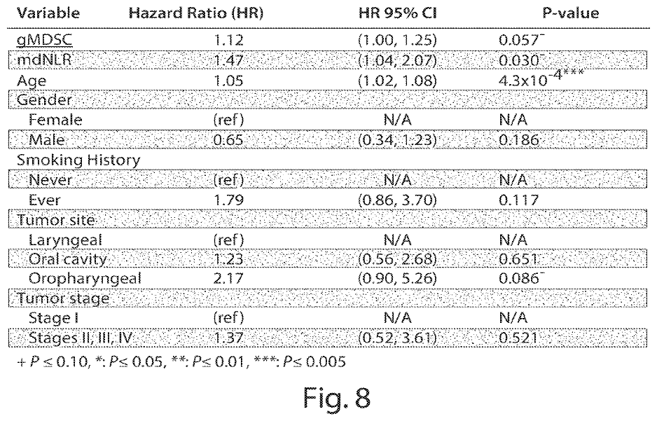

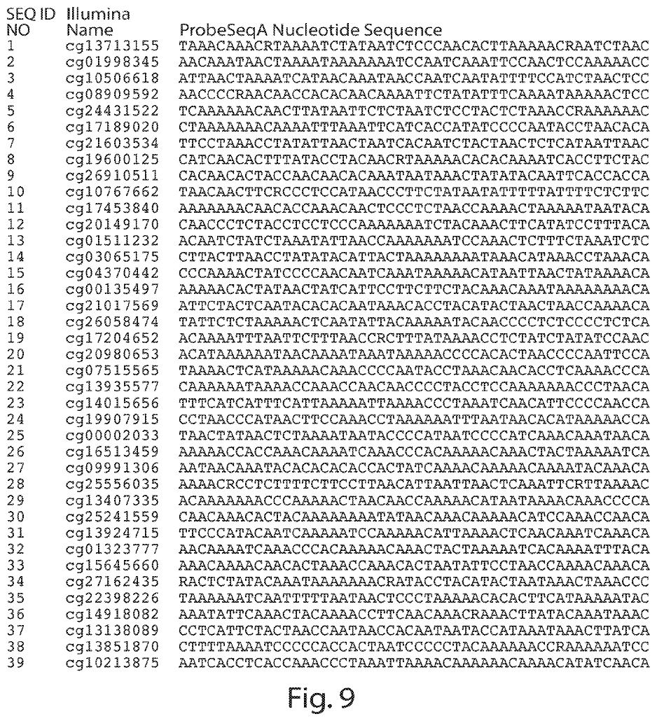

Candidate loci representing myeloid-specific CpGs were identified, and the top 100 included loci hypomethylated in myeloid cells compared to lymphoid cells and only a few loci that were hypermethylated in myeloid cells FIG. 6. Genes associated with these myeloid-specific loci are summarized in Table 6. Five loci were chosen that showed very strong correlation with the mdNLR across three independent blood DNA methylation datasets, FIG. 7. Among the different models examined, the quadratic form best fit the regression of CpG methylation and mdNLR. Table 7 describes the methylation levels of these five loci according to glioma patient characteristics (tumor grade, mutation status, NLR status). The data indicate the strong association of each individual loci with patient NLR status.

The performance of survival models that contain the mdNLR were compared, and a significant difference was observed from the base model which did not contain the mdNLR and a modest increase in the concordance score and Brier score (Table 5). Models that individually included one of each of the five myeloid-specific differentiation CpGs revealed that the loci were significant compared to the base model and produced concordance and Brier's scores equivalent to the mdNLR. As similar results were found when any of the five loci were included, only one of them (cg00901982) was included in Table 8. Also examined were models containing the mdNLR in addition to each of the five loci (Table 9). It was observed that if both variables are included in the models, little additional variance is explained.

TABLE 6
Top five myeloid-specific loci, SEQ ID NOs: 101-105

| | | | | | Gene located on | | | | |
|---|---|---|---|---|---|---|---|---|---|
| | Chromo- | MAPINFO hg19 | | Annotated | the same or opposite | SNP 10 bases | | Genomic | |
| | some | hg19 location | Strand | gene | transcription strand | to hybridization | MAF | context | Infinium |
| cg25938803 | chr2 | 43767347 | + | THADA | Opposite | rs183844032^{a} | 0.0002 | Body | II |
| Sequence^{b} | | | | | | | | | |

GCACTACAGCCAGTCACCAGCAATGACTGCAAGTAACTCTAGGACACTGACGCCTATTTGATTTGGAAGAGAATAAGGAACATAATGATGCCT
GAAATGTC

| | | | | | | | | | |
|---|---|---|---|---|---|---|---|---|---|
| cg00901982 | chr2 | 70257298 | − | PCBP-AS1 | Same | rs533928090 | 0.0002 | Body | II |
| Sequence^{b} | | | | | | | | | |

GACATTTCAGGCATCATTATGTTCCTTATTCTCTTCCAAATCAAATAGGCGTCAGTGTCCTAGAGTTACTTGCAGTCATTGCTGGTGACTGGCTG
TAGTGC

| | | | | | | | | | |
|---|---|---|---|---|---|---|---|---|---|
| cg01591037 | chr12 | 15134481 | − | PDE6H | Opposite | rs144778897^{b} | 0.001597 | 3UTR | II |
| Sequence^{b} | | | | | | | | | |

GACATTTCAGGCATCATTATGTTCCTTATTCTCTTCCAAATCAAATAGGCGTCAGTGTCCTAGAGTTACTTGCAGTCATTGCTGGTGACTGGCTG
TAGTGC

| | | | | | | | | | |
|---|---|---|---|---|---|---|---|---|---|
| cg10456459 | chr12 | 22843015 | + | ETNK1 | Same | rs373083641 | 0.0002 | 3UTR | II |
| Sequence^{b,c} | | | | | | | | | |

GCACTACAGCCAGTCACCAGCAATGACTGCAAGTAACTCTAGGACACTGACGCCTATTTGATTTGGAAGAGAATAAGGAACATAATGATGCCT
GAAATGTC

| | | | | | | | | | |
|---|---|---|---|---|---|---|---|---|---|
| cg03621504 | chr12 | 116571240 | + | MED13L | Opposite | N/A | N/A | Body | II |
| Sequence^{b} | | | | | | | | | |

GCACTACAGCCAGTCACCAGCAATGACTCAAGTAACTCTAGGACACTGACGCCTATTTGATTTGGAAGAGAATAAGGAACATAATGATGCCT
GAAATGTC
MAF minor allele frequency
ªSNP on hybridization site
^{b}Sequence corresponding to 50 bases upstream and 50 bases downstream of the CpG location based on the GRCh37/hg19 build; sequences and respectively SEQ ID NOs: 101-105
^{c}True enhancer

TABLE 7
NLR associated single CpG loci median (IQR) beta values in glioma patient subgroups

| | NLR status | | | | | | | | |
|---|---|---|---|---|---|---|---|---|---|
| | NLR | NLR | | Tumor grade | | | Mutation status | | |
| CpG loci | High | Low | P | II/III | IV | P | IDH1 | TERT | P |
| cg25938803 | 24.18 | 33.55 | <0.001 | 28.33 | 31.40 | 0.1 | 30.73 | 30.31 | 0.8 |
| | (22.72, 26.16) | (31.25, 39.00) | | (24.67, 32.50) | (26.20, 38.09) | | (26.13, 34.66) | (24.44, 35.24) | |
| cg00901982 | 20.63 | 30.29 | <0.001 | 24.19 | 27.71 | 0.04 | 26.71 | 24.69 | 0.5 |
| | (19.11, 22.61) | (26.67, 34.76) | | (21.17, 26.97) | (23.16, 34.32) | | (23.00, 30.73) | (21.12, 32.06) | |
| cg01591037 | 25.48 | 33.87 | <0.001 | 30.14 | 32.66 | 0.1 | 32.01 | 31.03 | 0.6 |
| | (23.58, 27.93) | (32.13, 39.76) | | (25.84, 33.75) | (27.94, 38.44) | | (27.50, 36.35) | (25.84, 36.50) | |
| cg10456459 | 26.49 | 38.02 | <0.001 | 32.19 | 36.44 | 0.05 | 36.14 | 32.97 | 0.5 |
| | (24.34, 28.20) | (35.68, 43.86) | | (27.47, 35.90) | (27.72, 44.06) | | (28.20, 39.10) | (27.36, 39.06) | |
| cg03621504 | 20.23 | 26.25 | <0.001 | 22.47 | 23.76 | 0.2 | 23.66 | 22.90 | 0.8 |
| | (18.28, 21.37) | (23.76, 30.42) | | (20.74, 26.12) | (21.39, 29.92) | | (20.89, 26.40) | (20.88, 26.56) | |

Note:
Beta values are represented as percentages (beta values times 100). Median differences between groups were tested using a Mann-Whitney U test

TABLE 8
Cox proportional hazards survival models including, age, sex, grade, mutation
status, and either mdNLR or cg00901982 (linear and quadratic terms

| | | | | | | Baseline + |
|---|---|---|---|---|---|---|
| | | | | | Baseline + | mdNLR + |
| | | | Baseline | Baseline + | mdNLR + | CpG + |
| | n | Mean | model HR | NLR HR | CpG HR | CpG² HR |
| | (%) | (sd) | (95% CI) | (95% CI) | (95% CI) | (95% CI) |
| Age | | 47 | 0.99 | 0.97 | 0.99 | 0.97 |
| | | (44, 54) | (0.94, 1.05) | (0.92, 1.03) | (0.94, 1.04) | (0.92, 1.03) |
| Female | 20 | | Referent | Referent | Referent | Referent |
| | (28) | | group | group | group | group |
| Male | 52 | | 0.75 | 0.74 | 0.74 | 0.69 |
| | (72) | | (0.41, 1.38) | (0.40, 1.35) | (0.40, 1.35) | (0.37, 1.26) |
| mdNLR > 4 | 28 | | | Referent | | |
| | (39) | | | group | | |
| mdNLR ≤ 4 | 44 | | | 0.49 | | |
| | (61) | | | (0.27, 0.90) | | |
| IDH only | 30 | | Referent | Referent | Referent | Referent |
| | (42) | | group | group | group | group |
| TERT only | 42 | | 3.96 | 4.56 | 4.25 | 4.65 |
| | (58) | | (1.98, 7.94) | (2.20, 9.43) | (2.09, 8.64) | (2.25, 9.62) |
| GBM | 33 | | Referent | Referent | Referent | Referent |
| | (46) | | group | group | group | group |
| 8Non-GBM | 39 | | 0.92 | 1.02 | 0.98 | 0.90 |
| | (54) | | (0.50, 1.71) | (0.54, 1.92) | (0.52, 1.82) | (0.48, 1.70) |
| cg00901982* | | 26.1 | | | 0.80 | 0.36 |
| | | (21.4, 31.2) | | | (0.52, 1.22) | (0.04, 3.15) |
| cg009019822* | | | | | | 29 |
| | | | | | | (3.52, 237) |
| Concordance | | | 0.71 | 0.73 | 0.72 | 0.74 |
| | | | (SE = 0.04) | (SE = 0.04) | (SE = 0.04) | (SE = 0.04) |
| Brier score | | | 0.1508 | 0.1506 | 0.1511 | 0.1468 |
| Lrtest vs baseline model | | | | 0.02 | 0.29 | 0.01 |
| Lrtest vs baseline + | | | | | <0.0001 | 0.06 |
| mdNLR model | | | | | | |
| Lrtest model linear (CpG) vs | | | | | | 0.01 |
| quadratic (CpG + CpG2) | | | | | | |
| model | | | | | | |

p values < 0.05 are highlighted in bold font
All covariates modeled met proportionality assumptions
HR hazard ratio,
CI confidence interval,
mdNLR methylation-derived neutrophil lymphocyte ratio,
Lrtest likelihood ratio test
*Per every 10% increase in methylation

TABLE 9
Cox proportional hazards survival models including, age, sex, grade,
mutation status, mdNLR and cg00901982 (linear and quadratic terms)

| | | | | | | Baseline + |
|---|---|---|---|---|---|---|
| | | | | | Baseline + | mdNLR + |
| | | | Baseline | Baseline + | mdNLR + | CpG + |
| | n | Mean | model HR | NLR HR | CpG HR | CpG² HR |
| | (%) | (sd) | (95% CI) | (95% CI) | (95% CI) | (95% CI) |
| Age | | 47 | 0.99 | 0.97 | 0.97 | 0.97 |
| | | (44, 54) | (0.94, 1.05) | (0.92, 1.03) | (0.92, 1.03) | (0.92, 1.02) |
| Female | 20 | | Referent | Referent | Referent | Referent |
| | (27.8) | | group | group | group | group |
| Male | 52 | | 0.75 | 0.74 | 0.74 | 0.7 |
| | (72.2) | | (0.41, 1.38) | (0.4, 1.35) | (0.41, 1.36) | (0.38, 1.29) |
| mdNLR ≥ 4 | 28 | | | Referent | Referent | Referent |
| | (38.9) | | | group | group | group |
| mdNLR < 4 | 44 | | | 0.49 | 0.40 | 0.69 |
| | (61.1) | | | (0.27, 0.9) | (0.17, 0.92) | (0.26, 1.81) |
| IDH only | 30 | | Referent | Referent | Referent | Referent |
| | (41.7) | | group | group | group | group |
| TERT only | 42 | | 3.96 | 4.56 | 4.49 | 4.73 |
| | (58.3) | | (1.98, 7.94) | (2.20, 9.43) | (2.16, 9.35) | (2.26, 9.88) |
| GBM | 33 | | Referent | Referent | Referent | Referent |
| | (45.8) | | group | group | group | group |
| Non-GBM | 39 | | 0.92 | 1.02 | 1.00 | 0.92 |
| | (54.2) | | (0.50, 1.71) | (0.54, 1.92) | (0.53, 1.90) | (0.48, 1.76) |
| cg00901982* | | 26.1 | | | 1.20 | 0.91 |
| | | (21.4, 31.2) | | | (0.72, 1.99) | (0.03, 24.7) |
| cg009019822* | | | | | | 15.9 |
| | | | | | | (1.12, 225) |
| Concordance | | | 0.71(SE = 0.04) | 0.73(SE = 0.04) | 0.74(SE = 0.04) | 0.74(SE = 0.04) |
| Brier score | | | 0.1508 | 0.1506 | 0.1504 | 0.1473 |
| Lrtest vs baseline model | | | | 0.02 | 0.06 | 0.02 |
| Lrtest vs baseline + | | | | | 0.49 | 0.13 |
| mdNLR model | | | | | | |
| Lrtest model linear (CpG) vs | | | | | | 0.06 |
| quadratic (CpG + CpG2) | | | | | | |
| model | | | | | | |

p values < 0.05 are highlighted in bold font
All covariates modeled met proportionality assumptions
HR hazard ratio,
CI confidence interval,
mdNLR methylation-derived neutrophil lymphocyte ratio,
Lrtest likelihood ratio test
*Per every 10% increase in methylation

### Example 13. Human Head and Neck Squamous Cell Carcinoma

Head and neck cancers are appearing in increased frequency due to epidemic transmission of human papilloma virus infection, particularly strain16, associated with genital and oral transmission.

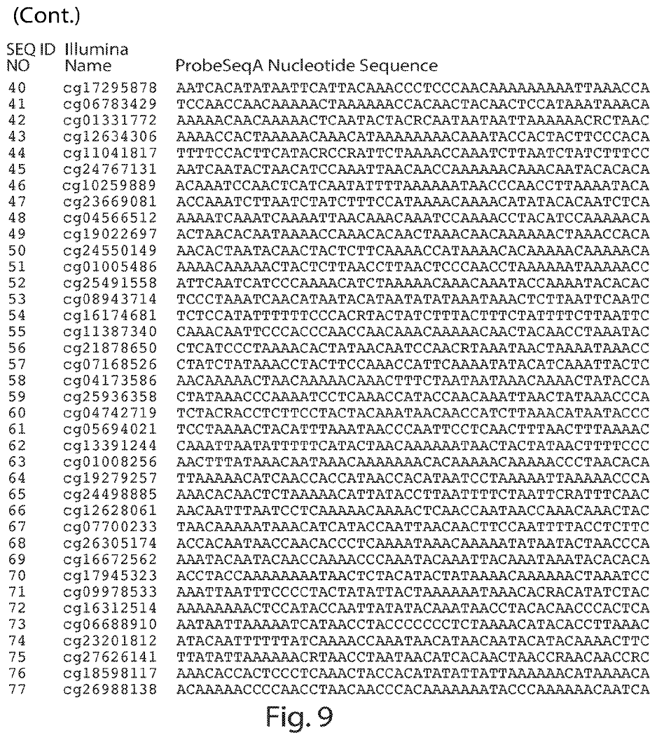

FIG. 8 shows Cox proportional hazards model of MDSCs (using the small 27K platform) predicting survival in head and neck cancer. Hazard ratios were elevated in patients in stages II, III and IV, in those with oropharyngeal tumors, and in smokers, compared with stage I cancer or non-smoker control patients. The Cox proportional hazards model demonstrates that an increased NLR and increased gMDSC proportion have statistically significant, independent associations with worse prognosis in head and neck cancer when adjusting for potential confounders age, gender, smoking history, tumor site, and tumor stage.

Shifts in the distribution and numbers of blood leukocytes as well as the emergence of aberrant myeloid cells with immunosuppressive properties are important predictors of cancer patient survival. See, Gabrilovich D I et al. Nat Rev Immunol. 9:162-74 (2009); Hagerling C et al. Trends Cell Biol. 25:214-20 (2015); and Parker K H et al. Adv Cancer Res. 128:95-139 (2015). The simple NLR in the whole blood has received attention as a replicated marker of cancer inflammation linked to poor survival, See, Templeton A J et al. J Natl Cancer Inst. 2014; 106(6): dju124 (2014). Because the NLR reflects the relative balance of the myeloid and lymphocytic lineages in peripheral blood, it is sensitive to the altered myelopoiesis arising in chronic inflammation and cancer.

A main finding of examples herein is that DMRs that distinguish leukocyte subtypes can be used to estimate the NLR ratio and that this epigenetically derived metric, like the cytological NLR, is associated with glioma occurrence and survival times. Although the mdNLR is less dramatically elevated in non-GBM compared with GBM cases, the data suggest alterations in some lower grade patients. The sample of glioma patients used herein was restricted to tumor subtypes containing either an IDH or a TERT mutation, exclusively. After adjustments for these molecular features and other prognostic factors, the elevated mdNLR determined to be observed herein was a significant prognostic indicator of shorter survival times. Thus, the immunomethylomic approach to the evaluation of the NLR holds considerable promise in immune profiling. Currently, there is intense interest in multiscale assessment of immune function in cancer patients receiving traditional treatments and new immunotherapies, See Blank C U et al. Science 352:658-60 (2016). Immunomethylomic methods herein can readily provide cell ratios as in the mdNLR and has the potential to identify aberrant epigenetic subsets of immune cells.

Evaluation of the performance of multivariate survival models with or without the mdNLR, yielded a significant improvement of model fit by inclusion of the mdNLR. The molecular subtypes selected for the current study represent very divergent prognostic groups. Survival for patients with IDH-only mutant glioma is much longer compared with those harboring TERT promoter mutation only tumors. Thus, survival models containing these mutation factors explain a large degree of variation in survival times and accordingly, improvements in predictive performance above the base model were modest in size, common to cancer studies. Nonetheless, the direction of the association of the mdNLR with survival is consistent with previous studies in glioma and other solid tumors that implicate myeloid factors in cancer inflammation. While the mdNLR is affected by either increased myeloid or decreased lymphocyte counts, the individual myeloid-specific differentiation loci are less susceptible to this effect of lymphocyte depletion. It is of interest therefore, that each of the five myeloid-specific loci performed similarly to the mdNLR and produced largely comparable performance metrics in multivariate analyses.

The current markers of leukocytes, mdNLR, and myeloid differentiation are easily implemented in clinical studies and large population studies. Unprocessed peripheral blood and archival samples are suitable for immunomethylomic profiling. The single CpG myeloid differentiation markers can be used in single locus quantitative assay formats without the requirement for extensive array-based analysis.

Ratio of neutrophils to lymphocytes (NLR) is here associated with immune suppression and decreased survival times in multiple solid tumors. Based on immune cell-specific DMRs and validated cell deconvolution algorithms, the NLR in blood from glioma patients was estimated and glioma patients had elevated mdNLR scores compared to controls. The patient mdNLR scores were increased in patients with grade IV tumors compared to grade II/III. High mdNLR scores were associated with shorter survival. Candidate single (myeloid-associated) gene loci that were highly correlated with the mdNLR were identified. Single myeloid differentiation loci provide a simpler and cheaper alternative to the mdNLR, which requires complex array data. Immunomethylomics are useful and more convenient than conventional cell analysis in profiling glioma risk and survival.

What is claimed is:

1. An array for determining methylation status of leukocyte types in a biological sample by analyzing methylation of a plurality of CpG dinucleotides in a plurality of genes of the sample, the array comprising:
a surface having a plurality of oligonucleotide probes with nucleotide sequences selected from at least one of the group of SEQ ID NO: 1-100, each probe attached at an addressable location on the surface, each probe hybridizes to a nucleotide sequence of a methylated form or an unmethylated form of a CpG dinucleotide in a sequence of a gene in the sample.

2. The array according to claim 1, further characterized as having:
at least 5 probes, at least 10 probes, at least 25 probes, at least 50 probes, or at least 100 probes; and/or,
additional oligonucleotide probes attached to the array containing CpG dinucleotides that optimally discriminate among leukocyte types according to methylation status of CpG dinucleotides in a gene of the leukocyte type, and/or further comprising control probes; and/or,
the additional oligonucleotide probes comprise SEQ ID NOs: 101-105; and/or
the oligonucleotide probes of SEQ ID NOs: 1-100 and/or the additional probes are selected to distinguish CpG methylation profile DNA sequences of at least two leukocyte types selected from the group of: myeloid-derived suppressor cells (MDSCs), granulocytic MDSCs (gMDSCs), mMDSCs, mast cells, basophils, neutrophils, eosinophils, monocytes, natural killer cells (NK), activated NK cells, NKT cells, Th17 T cells, megakaryocytes, erythrocytes, cytotoxic T cells, double positive T cells, T helper cells, Treg cells, and B cells.

3. A method of using an array to determine proportions in a biological sample of a subject of leukocyte types to prognose and/or diagnose a disease state in the subject, the method comprising:
analyzing extent of hybridization of patient sample DNA to each of a plurality of oligonucleotide probes, the probes being affixed to at least two surfaces for each of methylated and unmethylated CpG sequences and otherwise identical in nucleotide sequence, the plurality of the nucleotide sequences selected from at least one of the group of SEQ ID NO: 1-100, for determining methylation status of at least one CpG dinucleotide in the DNA of the sample;
comparing methylation status of the plurality of CpG dinucleotides analyzed in the patient sample to a DNA methylation reference library, to determine proportion of each leukocyte type in the sample;
displaying the methylation status of the plurality of hybridized genes in the sample in a graphical representation, thereby generating an image of the methylation profile (methylome) of the leukocyte types in the patient sample; and,
prognosing and/or diagnosing the disease state in the patient associated with the methylation status of CpG sites in leukocyte types, the disease state selected from a cancer, a cardiac condition, inflammation, an autoimmune disease, and infection/sepsis.

4. The method according to claim 3, the prognosing and/or diagnosing further comprising:
associating the methylation status of CpG sites in specific leukocyte types being above a pre-determined statistical threshold by determining a multivariate proportional hazards ratio equal to or greater than 1.0 as an indicium of a prognosis of an increased risk of death in the patient from the disease or as a diagnosis of the disease; or,
associating the proportions of specific leukocyte types above a pre-determined statistical threshold of a neutrophil to lymphocyte ratio (mdNLR) equal to or greater than 1.0, at least about 2.0 or at least about or greater than 4.0 as an indicium of a prognosis of an increased risk of death in the patient from the disease or as a diagnosis of the disease; or,
associating myeloid derived suppressor cell (MDSC), or gMDSC proportions in the sample as greater than or equal to a pre-determined statistical threshold of a multivariate proportional hazard value equal to or greater than 1.0, greater than 2.0, or at least about or greater than 2.5 as an indicium of a prognosis of an increased risk of death in the patient from the disease or as a diagnosis of the disease.

5. In a method of predicting a methylation class membership of leukocytes in a bodily fluid sample of a patient, the methylation class membership corresponding to an epigenetic signature of a plurality of leukocyte types, in which the method includes steps of measuring amounts of DNA methylation in each of a plurality of leukocyte type populations to determine differentially methylated regions (DMRs), ranking leukocyte DMRs for each leukocyte type according to statistical strength of association of each of at least one DMR with each leukocyte type, clustering samples in a training set using a defined number of highest ranked leukocyte DMRs to determine clustering solutions, a clustering solution corresponding to the methylation class membership, and predicting the methylation class membership for the leukocyte types within a testing set by applying the clustering solutions obtained from the training set to highest ranked leukocyte DMRs in the testing set, the predicted methylation class membership being determined by testing association of the predicted methylation class membership with the statistical discriminatory strength of the at least one DMR among the leukocyte types, the improvement comprising:
obtaining leukocyte methylation data of the sample using an array containing a plurality of nucleotide sequences each having a CpG site affixed to the array;
identifying statistically predictive subset DNA methylation libraries by scanning candidate sets of putative leukocyte-specific methylation markers to find sets of CpG sites that characterize each of the respective leukocyte types in the sample estimated by a cell mixture deconvolution;
constructing and evolving subset libraries of DMRs consisting of CpG sites differentially methylated among leukocyte types, by iteratively selecting subsets of DMRs at each iteration based on the statistical contribution of each DMR to methylation class membership prediction accuracy;
modifying a probability of selection of the DMRs at each iteration, the probability of selection of a CpG being modified proportional to contribution of the at least one DMR to methylation class membership prediction accuracy; and,
comparing the subset library of the patient DMRs sample to DMRs of a reference-based library of a plurality of control samples from a plurality of normal patients, to obtain a prognosis and/or a diagnosis of a cancer of the patient.

6. The method according to claim 5, the array for analyzing proportions of specific leukocyte types in the sample comprising at least one oligonucleotide selected from the group of nucleotide sequences of SEQ ID NO: 1-100, and the leukocyte types selected from at least one of: myeloid-derived suppressor cells (MDSCs), granulocytic MDSCs (gMDSCs), mast cells, basophils, neutrophils, eosinophils, monocytes, natural killer cells (NK), megakaryocytes, erythrocytes, cytotoxic T cells, double positive T cells, T helper cells, Treg cells, and B cells.

7. The method according to claim 5, the applying the subset library further comprising: calculating a multivariate proportional hazards ratio for the sample from the patient to assess the relationship of cancer prognosis and/or diagnosis with methylation status of the leukocyte composition.

8. The method according to claim 7, comparing further comprises obtaining the prognosis and/or diagnosis of cancer by selecting the leukocyte composition methylation status from the group of myeloid-derived suppressor cell (MDSC) methylation status and granulocytic myeloid-derived suppressor cell (gMDSC) methylation status.

9. The method according to claim 8, selecting the leukocyte composition methylation status from the group of myeloid-derived suppressor cell (MDSC) methylation status and granulocytic myeloid-derived suppressor cell (gMDSC) methylation status further comprises calculating the gMDSC multivariate proportional hazards ratio, which as equal to or greater than 1.0 is an indicium of a prognosis of an increased risk of death in the patient from the disease or is a diagnosis of the disease.

10. The method according to claim 7, further comprising associating the multivariate proportional hazards ratio of at least about 1.0, or at least about 2.0 with an indicium of about a two-fold increase in the risk of death in the patient from the cancer.

11. The method according to claim 7, further comprising adjusting the multivariate proportional hazards ratio for tumor histology status, gene mutation status, patient age, patient history, and patient gender status.

12. The method according to claim 7, further comprising selecting the CpG sites for inclusion in the statistically predictive subset library those CpG methylation patterns that indicate MDSCs or gMDSCs in the sample.

13. A method of obtaining selection probabilities of leukocyte differentially methylated regions (DMRs) for inclusion in a statistically predictive subset library of DMRs for predicting leukocyte type methylation class membership of leukocytes in a blood sample from a subject for prognosis and/or diagnosis of cancer in the subject, the method comprising:
constructing a candidate DMR search space to compare mean methylation values among leukocyte types by identifying CpGs that uniquely characterize each leukocyte cell type, and randomly assembling subset DMR libraries with CpGs that uniquely characterize the leukocyte cell types through multiple iterations;
estimating leukocyte cell compositions in the sample using the assembled subset DMR libraries and cell mixture deconvolution, and computing leukocyte ratios from the estimated leukocyte compositions of the sample;
assessing the accuracy of leukocyte cell composition estimates by comparing statistical differences among observed cell compositions obtained by at least one method selected from the group of: fluorescence-activated cell sorting (FACS) and complete blood cell counts (CBC), to predicted cell compositions obtained from cell mixture deconvolution of normal control samples, and implementing an iterative leave-one out procedure to assess individual contributions of each CpG to statistical prediction performance of the methylation class membership of the leukocytes, and further computing a dispersion separability criterion (DSC) score to assess a DMR subset power for discriminating among leukocyte types, to select CpGs, and updating subset DMR library selection probabilities by modifying the CpGs selected using the statistical prediction performance of a relative and of an absolute prediction accuracy of each CpG compared to remaining CpGs in the library, and using the updated probabilities in successive iterations to obtain updated probabilities, resulting statistically predictive subset DNA methylation libraries containing CpGs with the largest selection probabilities for improved accuracy of predicting leukocyte type methylation class membership; and,
fitting the multivariate proportional hazards ratio calculated from the sample to the updated subset DMR libraries thereby prognosing and/or diagnosing cancer in the blood sample from the subject.

14. The method according to claim 13, computing leukocyte ratios from the estimated leukocyte cell compositions further comprising comparing amounts of at least two different leukocyte types present in the leukocyte cell composition of the sample from the subject.

15. The method according to claim 13, the fitting the multivariate proportional hazards ratio further comprising comparing the hazard ratio to a Kaplan Meier plot of cancer survival data to prognose subject survival probability.

16. The method according to claim 14, further comprising calculating a neutrophil to lymphocyte ratio (mdNLR) and fitting the multivariate proportional hazards ratio to the mdNLR.

17. The method according to claim 13, the updated statistically predictive subset DMR library further comprising CpG sites of granulocytic myeloid-derived suppressor cells (gMDSCs) in the sample from the subject.

18. The method according to claim 13, the statistically predictive subset DMR libraries further comprising CpG sites the methylation status of which indicates MDSCs in the sample from the subject.

19. The method according the claim 13, the dispersion separability criterion (DSC) score defined as Db/Dw, wherein Db is a measure of dispersion between cell types and Dw is a measure of dispersion within cell types, and is implemented to quantify dispersion between leukocyte types and within leukocyte types for a randomly selected DMR subset.

20. The method according to claim 13, wherein the cancer is glioma or head and neck cancer.

21. A device having at least two surfaces each having an array comprising oligonucleotide of defined sequence each at an addressable location, the sequences selected from at least one of the group of SEQ ID NOs: 101-105.
